(19) United States
(12) Patent Application Publication
(10) Pub. No.: US 2007/0149881 A1
(43) Pub. Date: Jun. 28, 2007

(54) Ultrasonically Powered Medical Devices and Systems, and Methods and Uses Thereof

(76) Inventor: Barry Hal Rabin, Idaho Falls, ID (US)

Correspondence Address: BARRY H. RABIN, 3660 W. 81ST. SOUTH, IDAHO FALLS, ID, 83402, US

(21) Appl. No.: 11/615,570

(22) Filed: Dec. 22, 2006

Related U.S. Application Data
(60) Provisional application No. 60/753,447, filed on Dec. 22, 2005.
(60) Provisional application No. 60/806,542, filed on Jul. 4, 2006.

(51) Int. Cl. A61B 8/14

(52) U.S. Cl. 600/471

(57) ABSTRACT

The present invention provides a new family of ultrasonically powered medical devices and systems for powering such devices. Disclosed are methods for improving the overall power transfer efficiency of devices according to the present invention, as well as a wide variety of medical uses for such devices and systems. Devices of the present invention comprise a transducer that, during operation, converts electrical energy into high frequency, low amplitude mechanical vibrations that are transmitted to a driven-member, such as a wheel, that produces macroscopic rotary or linear output mechanical motions. Such motions may be further converted and modified by mechanical means to produce desirable output force and speed characteristics that are transmitted to at least one end-effector that performs useful mechanical work on soft tissue, bone, teeth and the like. Power systems of the present invention comprise one or more such handheld devices electrically connected to a power generator. Examples of powered medical tools enabled by the present invention include, but are not limited to, linear or circular staplers or cutters, biopsy instruments, suturing instruments, medical and dental drills, tissue compactors, tissue and bone debriders, clip appliers, grippers, extractors, and various types of orthopedic instruments. Devices of the present invention may be partly or wholly reusable, partly or wholly disposable, and may operate in forward or reverse directions, as well as combinations of the foregoing. The devices and systems of the present invention provide a safe, effective, and economically viable alternative source for mechanical energy, which is superior to AC or DC (battery) powered motors, compressed air or compressed gas, and hand powered systems.

### BACKGROUND OF THE INVENTION

1. Related U.S. Application Data

Provisional Application No. 60/753,447, filed Dec. 22, 2005 and Provisional Application No. 60/806,542, Filed Jul. 4, 2006.

2. Field of the Invention

The present invention relates to powered medical devices, systems for powering medical devices, and methods and uses of powered devices and power systems for a variety of medical purposes.

3. Description of the Prior Art

The use of medical and dental tools that utilize linear or circular motions to separate, attach, reshape, and remove soft-tissue, bone, teeth, and other types of living tissue is well known in the art. Medical drills for example are used in general and orthopedic surgeries, in common dental care, and in facial and other reconstructive procedures. Examples of other medical tools that utilize linear or circular motions include linear and circular staplers, linear and circular cutters, biopsy devices, suturing devices, drills, debriders, and tissue compactors. Linear and circular staplers and cutters utilize linear motions to form one or more lines of staples that attach two or more layers of tissue and can separate tissue layers in the center of the staple lines. Tissue compactors utilize circular motions to debulk removed tissue in order to enable passage of the removed tissue through narrow ports that are used for access in minimally invasive surgeries. Suturing devices utilize circular and linear motions to suture or attach various types of soft and hard tissue types. Biopsy devices utilize linear and circular motions to remove specific desired tissue samples and transport these samples to designated containers to be analyzed by pathologists.

All of the above mentioned medical and dental devices require a source of power in order to produce the necessary circular or linear motions. Various conventional methods for providing power to these devices have been utilized and such devices are well known in the art. Medical and dental drills commonly utilize electric motors, as exemplified by U.S. Pat. No. 4,705,038, U.S. Pat. No. 5,689,159 and U.S. Pat. No. 6,329,778, or mechanical motors energized by compressed air or compressed gas, as exemplified by U.S. Pat. No. 3,835,858, U.S. Pat. No. 4,109,735 and U.S. Pat. No. 7,008,224. Linear and circular staplers (and staplers that additionally contain cutters), utilize the surgeon or dentist supplied manual or hand power, as exemplified by U.S. Pat. No. 4,608,981 and U.S. Pat. No. 6,032,849, electric motors as exemplified by U.S. Pat. No. 5,954,259, U.S. Pat. No. 6,126,670 and U.S. Pat. No. 6,843,403, or mechanical motors energized by compressed air or compressed gas, as exemplified by U.S. Pat. No. 3,837,555, U.S. Pat. No. 4,349,028 and U.S. Pat. No. 5,397,046. In cases of medical or dental tools that use electric motors to generate circular or linear motions, either AC line power or DC battery power is utilized as the fundamental power source. In cases of medical or dental tools that make use of compressed air or compressed gas to generate circular or linear motions, either a compressor energized by AC line power or cartridges that contain pre-compressed air or pre-compressed gas are utilized as the fundamental power source.

Each of the above mentioned methods utilized to generate the power necessary to produce the desired circular or linear motions presents a set of technical limitations and other shortcomings, as explained below.

In the case of medical or dental tools that utilize electric motors that are energized by AC line power, or in the case of mechanical motors that are energized by compressors actuated by AC line power, significant disadvantages and limitations relate to the cost and complexity of such systems. For motors energized by AC line power or power supplies, a control circuit must be designed and provided to regulate the power delivered to the motor. These power supplies and the associated circuit boards, user interface, cabling, as well as the motors themselves, are complicated and expensive, provide difficulties for sterilization and are often not compatible with increasingly popular magnetic resonance imaging (MRI) diagnostics In the case of pneumatically driven mechanical motors, compressors must be supplied with adequate working pressure and airflow, and precision air motors designed to convert the pressurized airflow into useful mechanical energy can be very complicated and expensive. In both cases, these systems are further complicated and costs further increased because of the surgeon's need for instantaneous startup of the motor upon energizing and instantaneous stopping of the motor when power is turned off, which require additional design features to be added to the systems.

In cases of medical or dental tools that utilize electric motors energized by DC power sources such as batteries, one disadvantage and limitation includes the restricted electrical power available to such motors due to the size constraints of battery storage systems. Sterilization and shelf life considerations for battery powered systems further restrict device performance, and decreased battery reliability over time increases the risk of power loss during a medical procedure. When the batteries are made replaceable or rechargeable to circumvent some of the above limitations it unduly burdens the end user to maintain a ready supply of replacement batteries or separate charging systems for each device used, and to insure that the recharged battery is re-sterilized in preparation for its next use. These are significant limitations for battery powered systems.

In cases of medical or dental tools that utilize cartridges that contain pre-compressed air or pre-compressed gas, the disadvantages and limitations include pressure reduction within the pressure module over time, pressure fluctuations due to changes in ambient temperature, and safety risks such as the potential for high pressure leaks, the absence of pressure to actuate the device should a leak occur, and the associated surgical risks such as infection or failure to complete the procedure. The complexities and expense associated with ensuring integrity of the pneumatic path to prevent leaks and under-powering are significant drawbacks of these systems.

In cases of medical or dental tools that utilize surgeon or dentist supplied manual or hand-power a surgeon is required to pump a trigger or handle and the disadvantages and limitations include a lack of continuous hand power to effect the functional requirements of the device, inordinate levels of power required to effect actuation of the devices (which can be a significant disadvantage for physicians having limited hand strength), hand fatigue, unintended or secondary movements by the surgeon when attempting to actuate the device, and relatively long times required to actuate the devices.

Considering the technical limitations and shortcomings associated with the various methods utilized in prior art to energize and power medical and dental tools that require linear or circular motions, as described above, it is apparent that a safe, effective, and economically viable and readily available mechanical energy source could be most beneficial to patients, surgeons, dentists, and healthcare systems.

As will be described below, the present invention utilizes ultrasonic energy to overcome the above stated technical limitations and shortcomings. The use of ultrasonic energy in medicine is well known in the art. For example, ultrasonic imaging systems rely upon the transmission of ultrasonic signals to the body and subsequent recording of the reflected ultrasonic signals, followed by signal processing to generate a useful image of tissue. Exemplary prior art is disclosed in U.S. Pat. No. 5,740,128, U.S. Pat. No. 6,511,433 and U.S. Pat. No. 6,645,148.

Another common use for ultrasonic energy in medicine is the treatment of wounds or physical injuries, whereby ultrasonic energy is applied directly to the damaged tissue, most often transcutaneously, in order to generate a heating effect, increase blood flow or otherwise promote healing. Exemplary prior art is disclosed in U.S. Pat. No. 5,618,275, U.S. Pat. No. 6,685,656, U.S. Patent Application No. 20040171970A1.

Other common uses of ultrasonic energy are in dental tools and systems where ultrasonic vibrations are used for cleaning of teeth, roots, and debriding of bone in maxilo-facial procedures. For example, dental scalers are ultrasonic power systems commonly used in dental clinics, and ultrasonic toothbrushes are now widely used in the home. Exemplary prior art is disclosed in U.S. Pat. No. 5,150,492, U.S. Patent Application No. 20040023187A1, U.S. Patent Application No. 20050091770A1 and U.S. Patent Application No. 20050181328A1.

Other common uses of ultrasonic energy relate to therapeutic functions that rely on tissue effects such as ablation. Exemplary prior art is disclosed in U.S. Pat. No. 5,523,058, U.S. Pat. No. 6,126,619 and U.S. Patent Application No. 20040254569A1.

Another common use of ultrasonic energy is in general surgical procedures where ultrasonic vibrations are used for cutting and coagulation of blood vessels and soft tissue. Exemplary prior art is disclosed in U.S. Pat. No. 6,024,750, U.S. Pat. No. 6,036,667, U.S. Pat. No. 6,004,335 and U.S. Pat. No. 6,887,252.

In the above mentioned prior art where ultrasonic energy is used in surgical procedures for cutting and coagulation, ultrasonic power generators are used to supply the ultrasonic energy that is then transmitted to the treatment area. Such ultrasonic power generators are now widely available in surgical and dental facilities worldwide, as exemplified by commercial products such as the AutoSonix™ system by United States Surgical Corporation, the SonoSurg™ system by Olympus Surgical and Industrial America Inc., and the Harmonic™ system by Ethicon Endo-Surgery, Inc.

Regarding the prior art ultrasonic power systems used in surgical procedures for cutting and coagulation of tissue, or dental ultrasonic scalers used for cleaning teeth and bone, these systems generally consist of three main components: (1) an ultrasonic power generator (2) an ultrasonic transducer, typically embedded in a reusable handle held by the user and connected to the ultrasonic power generator by a cable, and (3) a plurality of instrument attachments, each containing an end-effector at the distal end that may be brought into contact with the target tissue, bone, or tooth in order to accomplish the desired medical or surgical effect. The ultrasonic power generator provides electrical signals that cause the ultrasonic transducer to resonate, thereby converting the electrical signals into high frequency, low amplitude (microscopic) mechanical vibrations that are operatively transmitted to the attached instrument and end-effector, which then also vibrates at high frequency and low amplitude. All of these prior art ultrasonic systems rely upon the generation, transmission, and application to the tissue of high frequency, low amplitude mechanical vibrations. At the tissue, for example, the frequency of vibration is typically in range of 20-200 kHz, the peak amplitude of vibration is typically in the range of 20-200 μm, and tip speeds are typically in the range of 2-20 m/s [1]. As a result, the mechanical forces generated by the devices on the tissue are limited, typically in the range of 0.1-1.0 N/mm. It is important to note that in all these prior art surgical devices, it is specifically the application of these high frequency, low amplitude mechanical vibrations directly to the target tissue that provides the medical effect and associated benefits.

There is considerable prior art involving the use of ultrasonic energy outside of the medical field. For example, one well developed area involves non-destructive testing or non-destructive evaluation, where ultrasonic energy, either transmitted or reflected, is used to inspect engineering structures for the presence of flaws or defects by employing imaging and signal processing methods [2, 3].

Another well established field involving ultrasonic energy relates to devices commonly known as ultrasonic (or piezoelectric) motors and actuators. Such motors and actuators have been explored for many years as potential alternatives to conventional electromagnetic motors [4, 5]. Exemplary prior art includes U.S. Pat. No. 4,019,073, U.S. Pat. No. 4,325,264 and U.S. Pat. No. 6,242,850, which are known as linear ultrasonic motors, and U.S. Pat. No. 4,484,099 and U.S. Pat. No. 5,336,958 which are known as traveling wave ultrasonic motors. In general, these ultrasonic motor and actuator technologies have achieved limited commercial success and are used in certain niche applications for micro-positioning and actuation, for example, in space exploration, electronics, optics, auto-focus cameras, automotive components, and the like, where small size, low power and high precision are required, or where special environmental considerations (e.g. vacuum or the presence of strong magnetic fields) preclude the use of conventional electromagnetic motors.

### BRIEF SUMMARY OF THE INVENTION

The present invention provides a new type of powered medical device, provides systems for powering a plurality of such devices, and discloses the use of these devices and systems for a wide variety of medical purposes. The present invention is based upon the conversion of high frequency, low amplitude mechanical vibrations generated by a transducer into macroscopic circular or linear motions, which are in turn converted by mechanical means into linear or rotary output motions having sufficient stroke, force, speed and precision to accomplish the desired medical tasks. Mechanical forces generated at the tissue by ultrasonically powered devices of the present invention far exceed anything possible with prior art ultrasonic surgical devices, thereby enabling a variety of medical mechanical procedures to be performed that were not previously not possible using ultrasonic energy sources.

In one preferred embodiment of the present invention, an ultrasonic power generator provides electrical signals to an ultrasonic transducer to produce the necessary high frequency, low amplitude mechanical vibrations. The basic principles employed to convert these mechanical vibrations into macroscopic mechanical motion are known in the art of ultrasonic motors and actuators. In the present invention, however, the mechanisms used to implement these principles have been uniquely adapted, combined with other mechanical elements, and configured in novel ways to create an entirely new class of powered medical devices that have unexpectedly been found to produce sufficient forces, speeds and other operating characteristics that are beneficial for a wide variety of medical purposes.

The devices and systems of the present invention, along with the methods and uses of these devices and systems disclosed herein, offer a number of unique advantages and overcome a number of important shortcomings and limitations of prior art powered medical devices. For example, devices of the present invention are simpler, smaller and less expensive to make and use, and are also easier and are more reliable to operate compared to prior art powered medical device technologies. This increases patient safety and lowers the overall cost of medical care. Further, compared to prior art powered medical devices, the devices of the present invention are uniquely capable of instantaneous startup and stopping when energized and de-energized, respectively, they hold fixed position and do not slip when de-energized, and are capable of generating significant mechanical forces that are substantially independent of the speed of actuation, all of which are uniquely beneficial features for many medical procedures. The devices of the present invention can be readily sterilized, and unlike conventional electromagnetic motors, they contain no magnetic components and are therefore completely compatible with MRI diagnostics. These unique features provide significant advantages over the prior art powered medical devices, especially for surgeons that are required to perform increasingly popular and precise minimally invasive endoscopic and laproscopic procedures. Additionally, ultrasonic power generators that may be readily used in systems of the present invention already exist in many surgical and dental facilities around the world, however their utility is currently limited to ultrasonic cutting and coagulation procedures and dental cleaning only. Therefore, by utilizing the devices and systems of the present invention, health care professionals that have previously purchased these expensive ultrasonic power generators will benefit from having a wider variety of medical uses for this equipment at their disposal, better justifying their initial capital investment.

Accordingly, it is evident that the devices and systems of the present invention provide a safe, effective, and economically viable alternative source for mechanical energy, which is superior to AC or DC (battery) powered motors, compressed air or compressed gas, and hand powered systems.

### BRIEF DESCRIPTION OF THE FIGURES

FIG. 1a illustrates the edge-drive linear friction principle employed to convert high frequency, low amplitude mechanical vibrations into macroscopic motion according to one embodiment of the present invention.

FIG. 1b illustrates the surface-drive linear friction principle employed to convert high frequency, low amplitude mechanical vibrations into macroscopic motion according to one embodiment of the present invention.

FIG. 1c illustrates a method of creating multiple points of contact between a single vibrating transducer and a driven member according to one embodiment of the present invention.

FIG. 1d illustrates a method of utilizing multiple vibrating transducers in contact with a single driven member according to one embodiment of the present invention.

FIG. 2 illustrates a system of the present invention wherein a plurality of powered medical devices of the present invention interchangeably connect to and are energized by a power generator.

FIG. 3 shows details of a handheld medical mechanical device according to one embodiment of the present invention.

FIG. 4 shows details of a device according to one embodiment of the present invention wherein high frequency, low amplitude mechanical vibrations are converted into macroscopic motion: (a) side view and (b) top view.

FIG. 5 is a schematic showing details of various possible surface modifications of a driven member wherein a non-smooth surface is provided to increase frictional traction and power transfer efficiency in devices of the present invention.

FIG. 6 shows details of the optional resonator according to one embodiment of the present invention.

FIG. 7 shows several possible methods for achieving selectable forward and reverse output mechanical motions in devices of the present invention.

FIG. 8 shows details of a handheld medical mechanical device according to one embodiment of the present invention configured to generate relatively low speed, relatively high force linear output.

FIG. 9 shows details of a handheld medical mechanical device according to one embodiment of the present invention configured to generate relatively high speed, relatively low torque rotary output.

FIG. 10a shows the maximum linear output force for the functional prototype device of Example 1 at each of 5 different power levels when the gear ratio was 20:1.

FIG. 10b shows the maximum linear output force for the functional prototype device of Example 1 at each of 5 different power levels when the gear ratio was 100:1.

FIG. 10c compares the linear output speed of the device of Example 1 for gear ratios of 20:1 and 100:1.

FIG. 11a shows the output rotary power of the device of Example 2 at each of 5 different power levels.

FIG. 11b shows the output rotary linear speed and rpm of the device of Example 2 at each of 5 different power levels.

### DETAILED DESCRIPTION OF THE INVENTION

In systems of the present invention, a power generator is connected and supplies electrical energy to a transducer capable of converting the electrical energy into high frequency, low amplitude mechanical vibrations. In one preferred embodiment of the present invention the power generator is an ultrasonic power generator and the transducer is an ultrasonic transducer (also known as a piezoelectric transducer), however it will be recognized by those skilled in the art that other types of power generators and transducers, for example magnetostrictive power generators and magnetostrictive transducers, may also be used to generate substantially similar high frequency, low amplitude mechanical vibrations from electrical energy.

In devices of the present invention, the high frequency, low amplitude mechanical vibrations generated by at least one energized transducer are operatively transmitted by frictional contact, either directly or indirectly via an intermediate vibrating component, to at least one driven member capable of producing macroscopic output rotary motion, linear motion or any combination thereof. The output motions are then transmitted to, and used to drive, at least one end-effector disposed toward the distal end of the device in order to accomplish the desired medical tasks. The driven member may also be configured along with other mechanical elements as part of a larger driven mechanism to further convert the macroscopic output rotary or linear motion produced by the driven member into other output linear or rotary motions that are then used to drive the end-effector. The end-effector may be connected directly to the driven member, it may be connected to the larger driven mechanism, or alternatively, it may be configured toward the distal end of a separate instrument attachment that may contain additional mechanical elements that further convert the output mechanical motion to better accomplish the desired medical function.

In one embodiment of the present invention, the devices are designed to be used as handheld appliances that, when operating, are connected to the power generator by an electrical cable. When in use, the handheld appliance is therefore comprised of the transducer, the driven member and end-effector. The entire handheld appliance, or any of the individual components comprising it, may be provided sterile within sterile packaging and intended to be used on a single patient (i.e. disposable), or may be designed to be sterilized repeatedly for reuse on one or more patients. Each of the individual components comprising devices of the present invention may be provided as an integral portion of, or separable or detachable from, the other system components.

Briefly, therefore, medical devices according to one embodiment of the present invention comprise:
a) At least one transducer capable of converting electrical energy into mechanical vibrations;
b) At least one driven member in frictional contact with said at least one transducer, wherein during operation of said device said frictional contact between said at least one transducer and said at least one driven member produces output rotary motion, linear motion, or combinations thereof; and
c) At least one end-effector driven by said output rotary motion, linear motion, or combinations thereof.

Since a power generator is necessary to operate devices of the present invention, briefly therefore, systems of the present invention comprise:
a) A power generator;
b) At least one transducer capable of converting electrical energy into mechanical vibrations;
c) At least one driven member in frictional contact with said at least one transducer, wherein during operation of said device said frictional contact between said at least one transducer and said at least one driven member produces output rotary motion, linear motion, or combinations thereof; and
d) At least one end-effector driven by said output rotary motion, linear motion, or combinations thereof.

Ultrasonic generators according to the present invention have maximum power ratings preferably between 1 and 2000 Watts, more preferably between 10 and 1000 Watts, and most preferably between 20 and 500 Watts, with a frequency of operation between 1 and 500 kHz, more preferably between 10 and 250 kHz, and most preferably between 20 and 150 kHz. In one embodiment of the present invention, the power generator is energized by AC line power, and further incorporates a controller providing a means for displaying and variably controlling the output power. Examples of such controllers providing such variable control means include, but are not limited to, switches, knobs, triggers, foot pedals, wireless transmitters, voice activation, and the like.

Ultrasonic transducers of the present invention are of the types that are commercially available, typically comprising a stack of piezoelectric ceramic elements, for example lead zirconium titanate (PZT) or similar, capable of generating high frequency, low amplitude vibrations when energized with a high frequency alternating voltage and current. According to one embodiment of the present invention, the transducer is an assembly that further comprises one or more matingly connected metallic elements designed to reflect and amplify the high frequency, low amplitude mechanical vibrations toward the distal or output end of the transducer assembly.

In the case of standing wave-type transducers used in one embodiment of the present invention, a metallic end-element commonly known as the horn acts as an acoustic waveguide to focus and amplify the ultrasonic vibrations produced by the transducer, where the resulting vibrations are primarily longitudinal in nature. Ultrasonic motors made using this type of transducer, and that utilize primarily longitudinal vibrations, are commonly known as linear ultrasonic motors and are the simplest type of ultrasonic motor. When the total length of the transducer assembly, including the horn, is tuned to the target resonant frequency, when driven by the ultrasonic power generator the entire assembly resonates and becomes a source of standing acoustic waves, where the peak amplitudes of vibration are typically in the range of 1-500 μm. Typically the horn is made from precision machined high strength aluminum alloy or titanium alloy, which exhibit good acoustic properties, and it's length must be tuned carefully to match the operating frequency of the power generator. According to one embodiment of the present invention, a standing wave-type of transducer is used to produce low amplitude longitudinal vibrations with peak amplitudes of vibration most preferably in the range of 20-200 μm.

In the case of traveling wave-type transducers used in an alternative embodiment of the present invention, the transducer elements are configured, tuned and excited in such a manner as to focus and amplify the ultrasonic vibrations produced by the transducer assembly into a traveling wave-like motion, where primarily flexural vibrations are utilized. These transducers are used in traveling wave-type ultrasonic motors, and can also be placed in frictional contact with the driven members of the present invention.

It should be obvious to those skilled in the art that other types of ultrasonic transducers, utilizing other modes of vibration, can also be used in devices of the present invention. Examples of vibration modes that may be used in frictional contact with the driven members of the present invention include longitudinal vibrations, lateral vibrations, flexural vibrations, torsional vibrations, and combinations of the foregoing.

According to one embodiment of the present invention, the vibrating transducer assembly is placed in direct contact with a driven member in order to convert the high frequency, low amplitude mechanical vibrations into macroscopic output rotary motion, linear motion, or any combination of rotary and linear motion. Alternatively, contact between the transducer and the driven member may be made indirectly using an intermediate vibrating component. In one preferred embodiment of the present invention, indirect contact is made using an optional resonator component that, during operation of the device, is matingly connected to the transducer and which to acts as an intermediate acoustic waveguide, focusing and transmitting the high frequency, low amplitude ultrasonic vibrations from the transducer to the driven member. The optional resonator component must also exhibit good acoustic properties and is therefore typically manufactured using similar materials and methods, and may be constructed or configured as an extension of, the transducer assembly. The use of the optional resonator component allows for optimizing the acoustic amplification and vibration characteristics needed to achieve efficient power transfer to the driven member, and provides additional design flexibility for positioning and optimizing the frictional contact between the transducer and driven member.

According to the present invention, during operation of the devices, the driven member brought into frictional contact with the vibrating transducer or vibrating optional resonator provides the mechanical means capable of generating useful output circular motions, linear motions, or combinations thereof. Driven members of the present invention may have many different shapes and the surface that makes frictional contact with the vibrating element may therefore be a curved surface, a flat surface, or combinations of curved and flat surfaces. Examples of driven members that may be used include wheels, gears, belts, linear bars, rings, arc segments, cams, linkages, and the like, as well as combinations of the foregoing. In one preferred embodiment of the present invention, the driven member is a wheel that is fixedly mounted on a shaft or axle that is capable of rotating about its axis. Driven members of the present invention may be constructed of common metals or alloys such as steel, brass, aluminum, titanium, and similar, or they may alternatively be constructed of ceramics, plastics, composites, and the like, or any combination of the foregoing. In one embodiment of the present invention, the driven member is constructed of a material that has a higher hardness than the material used to manufacture the vibrating transducer assembly or optional resonator to which it makes frictional contact during operation. In a preferred embodiment of the present invention, the driven member is constructed of hardened steel or ceramic.

According to one embodiment of the present invention, the driven member is configured as part of a larger driven mechanism, said driven mechanism further comprising other mechanical elements that convert the macroscopic motion generated by the driven member into more desired output mechanical motions. In one embodiment of the present invention the output mechanical motion is a rotary or circular motion. In another embodiment of the present invention the output mechanical motion is a linear motion. Combinations of linear and rotary output mechanical motions are also possible.

In one preferred embodiment of the present invention the driven mechanism comprises a driven member that is a wheel mounted on a shaft or axle that is capable of rotating about its axis, and further comprises additional gear elements and shafts to adjust and control the speed and force of the linear or rotary output mechanical motion. As will be obvious to those skilled in the art, additional gears, shafts, transmissions, linkages, clutches, couplings and the like may be optionally included in the driven mechanism to further convert and optimize the driven member output mechanical motion to have the force, speed and other operating characteristics desired for the intended medical purpose. The driven mechanism of the present invention may be provided as one or more assemblies or subassemblies that may further comprise various other electronic, magnetic or electromechanical elements designed to improve the performance and enhance functionality, safety or control. Examples of such other elements include indicators, switches, actuators, fuses, circuits, microprocessors, and the like.

According to the present invention, a plurality of end-effectors may be either singly or interchangeably connected to, and are driven by, the driven member or driven mechanism. During operation, the end-effector may further convert or modify the output mechanical motions, and transmits said motions to the target tissue to effectively utilize the output mechanical motions for the purpose of performing medical work. Examples of such end-effectors include, but are not limited to, linear staplers, linear cutters, circular staplers, circular cutters, biopsy instruments, suturing instruments, medical and dental drills, tissue compactors, tissue and bone debriders, clip appliers, grippers, extractors, and various types of instruments used in orthopedic surgery. It is to be understood within the context of the present invention that the end-effectors disclosed herein are included for illustration and explanation purposes, and are not to be considered as limiting the scope of the present invention with regard to the type of medical procedures, functions, effects, or uses of the mechanical work that may be performed upon tissue, bone, teeth, and the like. During operation of devices of the present invention, the end-effectors may be directly connected to the driven member or they may be connected indirectly via a driven mechanism. Further, the end-effector may be configured within a larger instrument attachment, wherein said instrument attachment either connects directly to the driven member, or indirectly via a driven mechanism, and where the end-effector is disposed toward the distal end of said instrument attachment.

As will be obvious to those skilled in the art, additional gears, shafts, transmissions, clutches, linkages, couplings and the like may be optionally included in the instrument attachment to further convert and optimize the output motion generated by the driven member or driven mechanism to produce the force and speed characteristics desired for the intended medical purpose. The instrument attachments of the present invention may be provided as one or more assemblies or subassemblies that may further comprise various other electronic, magnetic or electromechanical elements designed to improve the performance and enhance functionality, safety or control. Examples of such other elements include indicators, switches, actuators, fuses, circuits, microprocessors, and the like.

According to the present invention, the various individual components comprising the devices and systems may be configured to be matingly connected, joined together, and assembled or disassembled, both in manufacturing and during medical use, by any connection methods commonly known to those skilled in the art of electromechanical assemblies and medical devices. Examples of such methods include but are not limited to plug connections, pin connections, screw connections, press-fit connections, adhesive connections, snap connections, spring connections, flange connections, bayonet connections, and the like.

In one preferred embodiment of the present invention the entire handheld portion of the medical device, comprising the transducer, driven member and end-effector, is designed to be reusable, being provided as a unitary structure that is capable of undergoing repeated sterilization treatment prior to reuse on one or more patients.

In another embodiment of the present invention the entire handheld portion of the medical device, comprising the transducer, driven member and end-effector, is designed to be disposable, being provided sterile within sterile packaging and intended to be used on a single patient.

In still other embodiments of the present invention the various components and subassemblies comprising the medical device may be designed and intended to be either reused on one or more patients or disposed of after use on a single patient. Further the various components and subassemblies comprising the medical device may be provided either as an integral portion of, or separable or detachable from, other system components. For example, according to one preferred embodiment of the present invention a medical device comprises a first component further comprising a reusable handle containing the transducer, and a second component, detachable from the first and that may be either reusable or disposable, said second component further comprising the driven member and end-effector.

According to yet another preferred embodiment of the present invention, the medical device comprises a first component, further comprising a reusable handle containing the transducer, a second component, detachable from the first and that may be either reusable or disposable, said second component further comprising the driven member, and a third component, detachable from the second and that may be either reusable or disposable, said third component further comprising at least one end-effector. It will be obvious to those skilled in the art that other configurations involving unitary vs. detachable components, as well as reusable vs. disposable components, are possible within the broad scope of the present invention. Such alternative embodiments provide added flexibility according to the different needs and desires of the device manufacturer or medical professional.

While the present invention will be described more fully hereinafter with reference to the accompanying drawings, in which particular embodiments are shown and explained, it is to be understood that persons skilled in the art may modify the embodiments herein described while achieving the same functions and results. Accordingly, the descriptions that follow are to be understood as illustrative and exemplary of specific structures, aspects and features within the broad scope of the present invention and not as limiting of such broad scope. Further, the methods and uses discussed herein shall not be construed as limiting the scope of the invention with regard to specific medical procedures or surgical applications, as they are only used as elucidating examples in which the present invention may be employed.

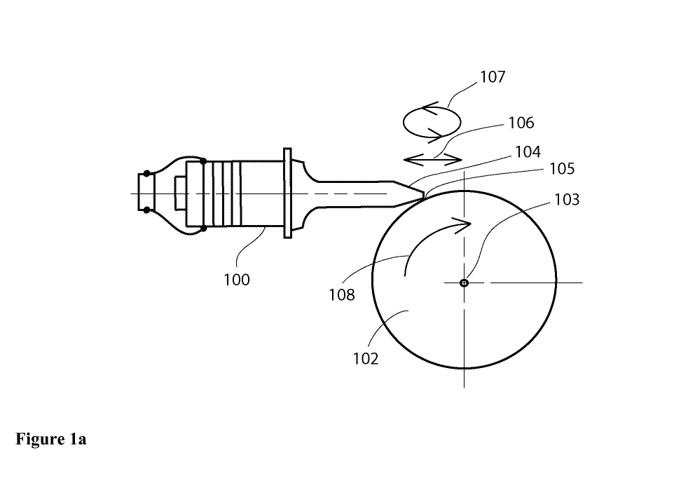

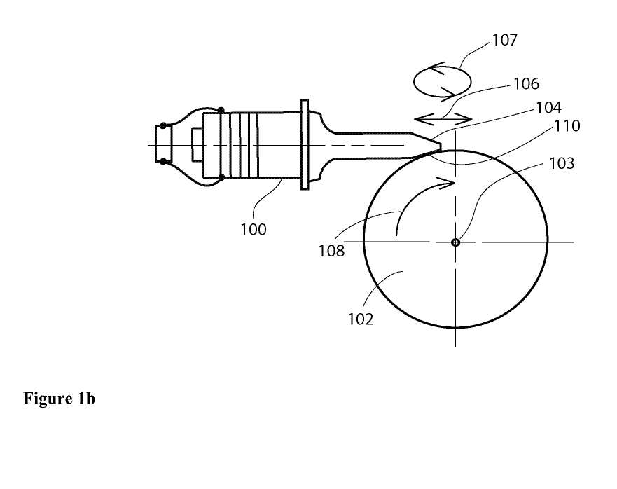

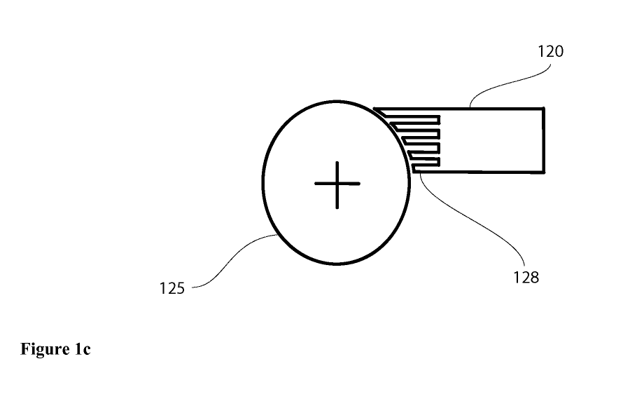

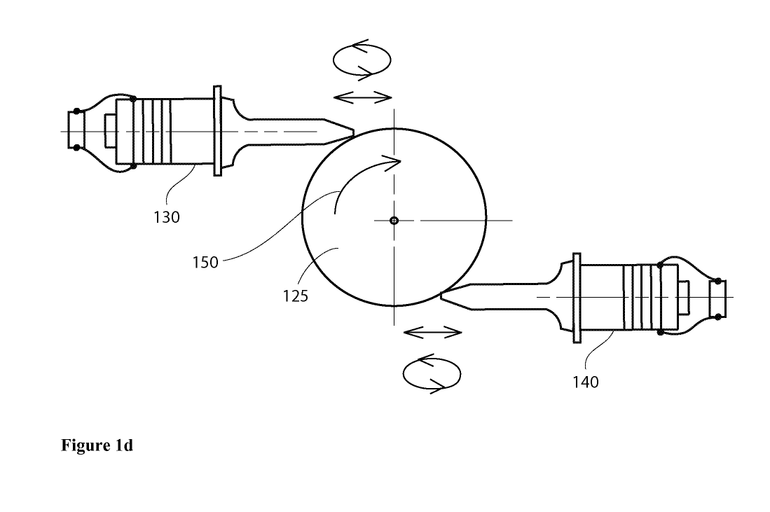

According to one embodiment of the present invention, the basic principles employed to convert high frequency, low amplitude mechanical vibrations generated by a standing wave-type transducer into macroscopic rotary motion are shown schematically in FIG. 1. In one embodiment, FIG. 1a, an edge-driven mode of operation is illustrated wherein transducer 100 is connected to a power generator (not shown) and is placed in frictional contact with wheel 102 (the driven member in this example) that is fixedly mounted onto shaft 103. Power transfer is made by bringing the distal tip of the transducer horn 104 into frictional contact with wheel 102 along edge 105. Upon energizing of the power generator, transducer 100 produces high frequency, low amplitude longitudinal vibrations 106, causing transducer horn tip 104 to repeatedly impact and rebound off of driven wheel 102. As a combined result of the longitudinal vibrations within transducer 100, the repeated impact and rebound between the distal tip of tranducer horn 104 and driven wheel 102, and the frictional forces that occur at the region of contact (edge 105), transducer horn tip 104 moves in an elliptical fashion 107, and wheel 102 rotates in a forward (clockwise) direction 108, thereby also causing shaft 103 to turn in a clockwise fashion.

In another embodiment of the present invention, FIG. 1b, a surface-driven mode of operation is illustrated, wherein distal tip of the transducer horn 104 is brought into frictional contact with wheel 102 along surface 110. Otherwise, the operation and method of power transfer in FIG. 1b is substantially equivalent to the description for FIG. 1a. It has been found by experiment with devices of the present invention that the surface-driven mode (FIG. 1b) is a preferred mode of operation that provides longer lifetime and greater reliability, explained as follows. During operation, the frictional interaction that occurs at the region of contact (surface 110 in FIG. 1b vs. edge 105 in FIG. 1a) results in wear and the gradual removal of material from the transducer. In the edge-driven mode of operation (FIG. 1a), the removal of material from the region of contact (edge 105) causes a reduction in the overall length of transducer 100 that eventually causes the transducer length to fall outside of the proper tuning range for efficient operation. In contrast, in the surface-driven mode of operation (FIG. 1b), the removal of material from the contact region (surface 110) does not cause the length to transducer 100 to change, which means the transducer remains properly tuned and operating efficiently for a longer period of time.

It is a claimed feature and benefit of the devices and systems according to the present invention that the output performance be optimized by maximizing the power transfer efficiency during the conversion from high frequency, low amplitude vibrations to medically useful output mechanical motions. Accordingly, and considering the basic principles for converting such high frequency, low amplitude vibrations as illustrated in FIG. 1, it has been found that numerous factors may be controlled and adjusted to improve the efficiency of power transfer to the driven member. The power rating, frequency range and other design features of the generator and transducer circuit are important to ensure proper and safe operation. The material selection, geometry and proper tuning of the transducer assembly or optional resonator components are critical for efficient generation of the needed high frequency, low amplitude vibrations. Other factors that have been found to be important for optimizing the power transfer efficiency include, but are not limited to, the mode of contact between the transducer (or optional resonator) and the driven member (i.e. edge-type or surface-type contact, according to FIG. 1a or FIG. 1b, respectively); the size, shape, material properties and surface characteristics of the transducer horn or optional resonator; the size, shape, material properties and surface characteristics of the driven member; the angle of contact, contact area and applied forces at the region of contact between the transducer or optional resonator and the driven member; and any other factors that influence the frictional interaction that occurs between the transducer or optional resonator and the driven member during operation. It has been found by experiment that of particular importance are the relative hardness and deformation characteristics of the frictionally contacting materials, as well as the surface condition of the driven member. These teachings will be discussed in greater detail below.

It is important to point out that while FIG. 1a and FIG. 1b schematically suggest a single transducer or optional resonator in contact with a single driven member, the present invention is not limited to such scope. For example, as shown in FIG. 1c, in order to increase the output performance and power transfer efficiency of the devices, there may be multiple points of contact established between a single transducer 120 and driven wheel 125, for example, by utilizing finger projections 128. As shown in FIG. 1d, there may be multiple transducers, such as first transducer 130 and second transducer 140 contacting the same driven wheel 125. In this embodiment of the present invention, it may be beneficial to configure two or more standing wave-type transducers on opposite sides of driven wheel 125, as shown, such that they reinforce the same direction of rotation 150. It may be additionally advantageous in such a configuration to control the phase relationship between the elliptical motion of the more than one vibrating transducers such that they are vibrating out of phase with each other, i.e. when one transducer is impacting the wheel the other is rebounding off of the wheel. By having the frictional impact events occur at alternating times, the driven member is powered more smoothly and continuously, resulting in greater power transfer efficiency.

As will be obvious to those skilled in the art, other configurations are also possible. For example, in another embodiment of the present invention (not shown), there may be more than one transducer and driven member subassemblies powering the same driven mechanism within a given medical device. According to another embodiment of the present invention (not shown), multiple points of contact between a single transducer and driven member may be accomplished utilizing a traveling wave-type of transducer assembly wherein the vibrating element attached to the transducer is in the form of a disk, sheet, ring, or similar shape. As a result of the flexural vibrations produced by these traveling wave-type transducer assemblies, more than one vibration amplitude peak exists that can therefore make multiple points of contact with a single driven member surface brought into frictional contact with said traveling wave-type of transducer.

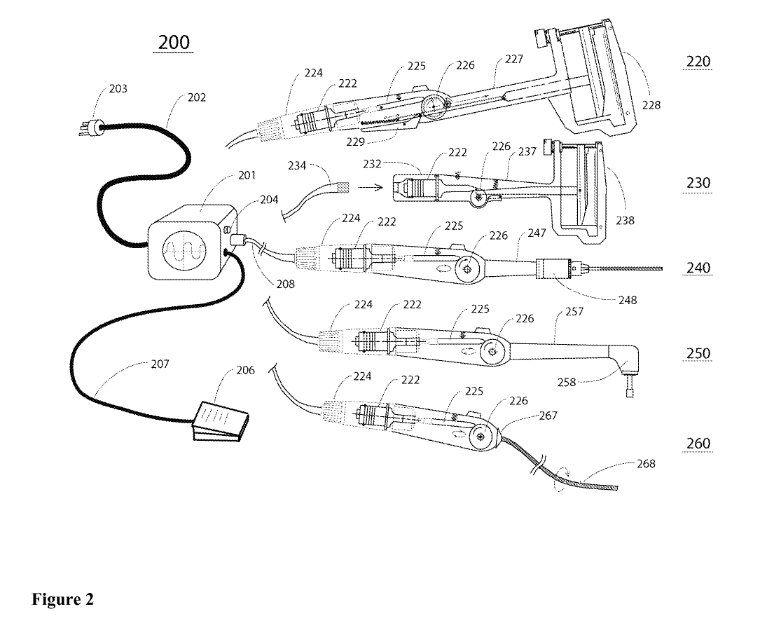

FIG. 2 schematically illustrates an integrated power system and plurality of medical devices according to one preferred embodiment of the present invention. Shown are a variety of handheld medical devices that, during operation, matingly and interchangeably connect to, and are powered by, an ultrasonic power generator. System 200 comprises an ultrasonic power generator 201 connected to the AC line-power via power cable 202 and power plug 203, having a main power switch 204, a foot activation switch 206 that is connected to said ultrasonic power generator via cable 207, and cable 208 that connects the power generator to the handheld devices. Examples of handheld devices provided for purposes of illustration include surgical staplers 220 and 230, a surgical or dental drill 240, a surgical or dental debrider 250, and a flexible rotating shaft surgical tool 260.

Each of the handheld medical mechanical devices of system 200 further comprises a transducer 222 embedded within a handle 224, optional resonator 225, a driven member 226, and a specific instrument attachment (227, 237, 247, 257, 267) with end-effector (228, 238, 248, 258 and 268) needed to accomplish the desired medical function, namely surgical stapler 220 and 230, surgical or dental drill 240, surgical or dental debrider 250, and flexible rotating shaft surgical tool 260, respectively. Note that in surgical stapler 220, a squeezable trigger or handle 229 is further provided and may serve one or more functions. In one embodiment of the present invention, squeezable trigger 229 provides a controlling means for disengaging the driven mechanism, allowing the instrument attachment to retract to its original position via an embedded spring (not indicated). In another embodiment of the present invention, squeezable trigger 229 provides an alternative and sometimes more convenient controlling means compared to foot activation switch 206 for activating, de-activating and controlling the level of power to the device or its output speed.

During operation of system 200, when power generator 201 is energized and the operator activates foot switch 206 (or squeezable trigger 229), electrical energy is transmitted to the transducer within the reusable handle, which generates high frequency, low amplitude mechanical vibrations. The high frequency, low amplitude mechanical vibrations are transmitted either directly or indirectly via optional resonator 225 to the driven member 226, that converts the motion into macroscopic rotary or linear output mechanical motions appropriately optimized in terms of speed, stroke, force and other characteristics for use in the intended medical procedure. The macroscopic rotary and/or linear mechanical motions output by driven member 226 are further converted and modified by mechanical means within the instrument attachments 227, 237, 247, 257, and 267, and are then transmitted to and drive the end-effector, namely 228, 238, 248, 258 and 268, which is the distal portion of the instrument attachment where medical work on tissue, bone or teeth, and the like, is actually performed.

Device 230 provides an example of a handheld mechanical device according to one embodiment of the present invention, in this case also a surgical stapler, where the entire handheld portion of the device 232, which comprises transducer 222, driven member 226, instrument attachment 237, and end-effector 238 is designed to be provided sterile in a sterile package and intended to be disposed of after initial use on a single patient. In device 230, cable 208 that supplies electrical signals from the ultrasonic generator connects to the disposable handheld device 232 using cable connector 234.

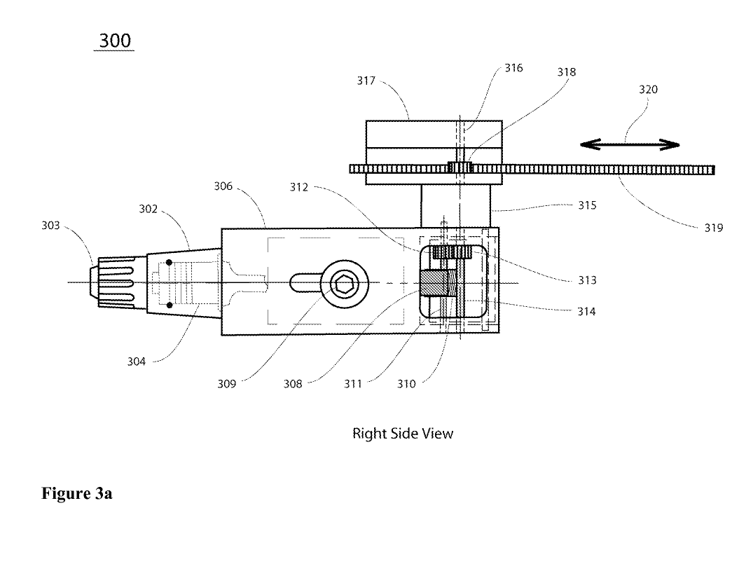

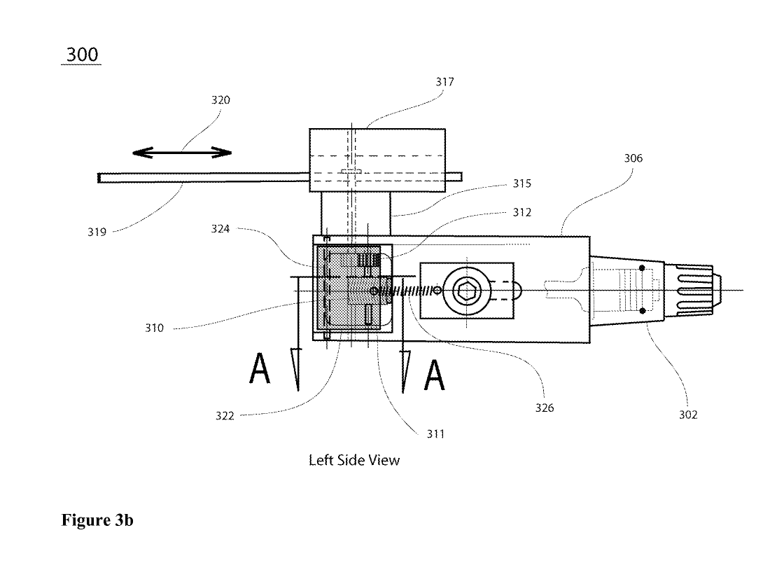

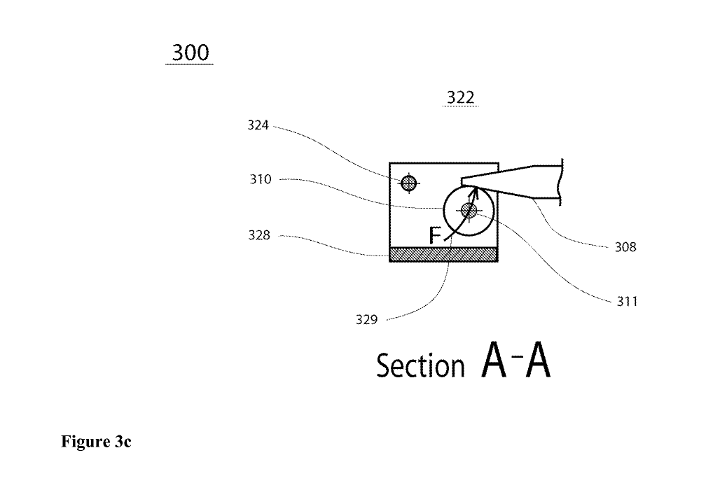

FIG. 3 shows a functional prototype of a handheld device 300 according to one embodiment of the present invention. To highlight some specific elements of this preferred embodiment, the right side view of device 300 is shown in FIG. 3a. Handpiece 302 that connects to the power cable (not shown) at connector 303 and contains transducer assembly 304 is removably attached to device body 306. Device body 306 also contains optional resonator 308, that is held in place and adjusted in position using adjustment bolt 309 relative to the position of driven wheel 310. Driven wheel 310 is fixedly mounted onto wheel shaft 311, onto which is also fixedly mounted driven gear 312. Driven gear 312 engages primary gear 313, that is fixedly mounted onto primary drive shaft 314. Primary drive shaft 314 engages with transmission 315, that further engages output drive shaft 316, that is mounted into output assembly 317 and onto which is fixedly mounted output gear 318. Output gear 318 engages rack 319 that moves in a forward and reverse linear fashion as shown in 320.

To highlight other specific elements of this preferred embodiment, the left side view of device 300 is shown in FIG. 3b. In this view, it can be seen that wheel shaft 311 (onto which is fixedly mounted driven wheel 310 and driven gear 312) is mounted within moveable carriage assembly 322, that is further mounted into device body 306 on rotatable shaft 324 such that the driven wheel 310 may pivot relative to optional resonator 308 to which it makes frictional contact during operation. Spring component 326 is mounted between device body 306 and moveable carriage assembly 322 such that it causes a known compressive force to be applied at the contact region between driven wheel 310 and optional resonator 308.

Driven wheel 310, together with the associated shafts, gears, transmission, etc. (i.e. elements 311-326 in FIG. 3) comprise a driven mechanism of the present invention in the example shown. End-effectors of the present invention (or instrument attachments further comprising said end-effectors), not shown in FIG. 3, matingly attach to the distal end of output assembly 317 and further convert and transmit forward and reverse linear output mechanical motion 320 of rack 319 into useful work for performing medical functions.

To further illustrate an important teaching according to devices of the present invention, FIG. 3c provides a detailed schematic view at section A-A indicated in FIG. 3b showing the relationship between optional resonator 308, driven wheel 310 and the function of moveable carriage assembly 322. Moveable carriage assembly 322 comprises bracket 328 mounted on rotatable shaft 324. Mounted within bracket 328 is wheel shaft 311 (onto which is fixedly mounted driven wheel 310 and driven gear 312). Spring component 326 (not shown) that is attached between bracket 328 and device body 306 causes moveable carriage assembly 322 to pivot on rotatable shaft 324, thereby applying force 329 at the region of contact between driven wheel 310 and optional resonator 308. The function of moveable carriage assembly 322 along with spring component 326 is critical for controlling the optimum force and angle of impingement of driven wheel 310 on optional resonator 308, even as dimensional changes caused by wear of the frictional components takes place during continued operation. This type of configuration, of which other variations may be obvious to those skilled in the art, ensures consistent device performance and power transfer efficiency throughout the device lifetime. In the configuration shown, force 329 is preferably between 0.01 kg and 10 kg, more preferably between 0.1 kg and 5 kg and most preferably between 0.2 kg and 2.5 kg.

Referring to FIG. 3, it is often advantageous in this type of device that handpiece 302 contains the transducer assembly and is provided as a component of the system that is removable and reusable (i.e. it can be repeatedly sterilized and used on one or more patients). In order to minimize acoustic losses it is important that handpiece 302 attach to the device in such a manner that the distal end of transducer assembly 304 comes into intimate mating contact with optional resonator 308, which is held in position within device body 306. This can be accomplished using various connection methods known to those skilled in the art. In one preferred embodiment, the distal end of transducer assembly 304 is threaded and screws together with optional resonator 308 at its proximal end. The location of optional resonator 308 within device body 306 is facilitated by adjustment bolt 309, which can be loosened to allow optional resonator 308 to slide forward and backward, and then tightened to hold optional resonator 308 in position. This allows positioning of optional resonator 308 relative to driven wheel 310 in order to establish the optimum frictional contact (i.e. edge-driven type vs. surface-driven type, according to FIG. 1). Transmission 315, located between primary drive shaft 314 and output drive shaft 316, provides the desired gear reduction and thereby substantially controls the output speed and force characteristics used to drive the end-effector.

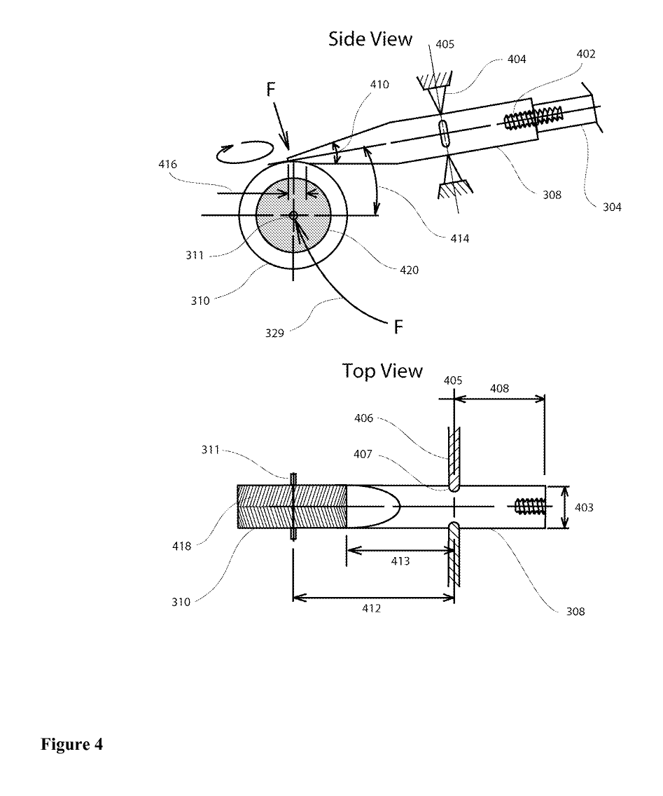

FIG. 4 schematically shows a close up view of the interior of device 300 wherein optional resonator 308 has a tapered distal end and is in frictional contact with driven wheel 310. The proximal end of optional resonator 308 is connected to the distal end of transducer assembly 304 via threaded connection 402. Optional resonator 308 is typically held in place within the device body (not shown) by mechanical supports 404. As is well know in the art, in order to maintain proper tuning of the acoustic resonator and avoid undesirable energy losses, such mechanical supports 404 are preferably located precisely at the position of an acoustic node 405, which is at a location along the length of resonator 308 where the displacements of the standing longitudinal acoustic wave pass through zero amplitude. In one embodiment of the present invention, illustrated in the top view in FIG. 4, optional resonator 308 having diameter 403 is held in place by pin-type supports 406 that engage within grooves 407 at acoustic node 405. Distance 408 between the proximal end of optional resonator 308 and acoustic node 405 is therefore established by the location of the acoustic node. Distance 413 between acoustic node 405 and the distal end of optional resonator 308 is also critical, and is preferably selected such that the distal end of optional resonator 308 (or more importantly, the location where optional resonator 308 makes contact with driven wheel 310), occurs at a location along the length of optional resonator 308 where the displacements of the standing longitudinal acoustic wave are large, preferably where the displacements of the standing longitudinal acoustic wave pass through maximum amplitude. Accordingly, both distances 408 and 413 are critical dimensions determined by the resonant frequency, size, shape, properties of the resonator and support materials, and other factors, such that the optional resonator is properly tuned and undesirable energy losses are minimized.

Numerous other factors may be optimized in devices of the present invention to increase the output performance, improve power transfer efficiency, reduce noise, increase lifetime and reliability, or decrease manufacturing costs. For example, as shown in FIG. 4, the included angle 410 at the tapered end of the optional resonator, the distance 412 between acoustic node 405 and the centerline of driven wheel 310, and the angle of impingement 414, all affect the relative size and position of the region of contact 416 between optional resonator 308 and driven wheel 310, as well as the amplitude of the standing longitudinal acoustic wave and other frictional characteristics at the region of contact 416 between optional resonator 308 and driven wheel 310. For the configuration shown, the angle of impingement 414 is preferably between 0° and 90°, more preferably between 0° and 75°, and most preferably between 0° and 60°. In one preferred embodiment of the present invention, the surface of driven wheel 310 is modified to be non-smooth in order to enhance the frictional traction and power transfer efficiency between optional resonator 308 and driven wheel 310. In the example shown, the surface of driven wheel 310 is made non-smooth via textured finish 418. In another preferred embodiment of the present invention, driven wheel 310 may contain a core 420 made from a material having different properties, for example, an acoustically dampening material. Other acoustic dampening elements may be included at various locations within devices of the present invention to further reduce audible noise, such as sealed air spaces, foams, insulations, coatings, and the like.

As is known in prior art ultrasonic motors, and confirmed by experiment with devices of the present invention, certain combinations of materials used to manufacture the interacting frictional components (i.e. the transducer assembly or optional resonator and driven member) yield increased performance, improved efficiency, reduced noise, or increased lifetime and reliability. Accordingly, in one embodiment of the present invention, optional resonator 308 is produced from a metallic material that is acoustically efficient. The acoustic impedance of a material is defined as the product of the velocity of sound within the material and its density, and is a useful design parameter. In devices of the present invention, the vibrating components comprise materials having an acoustic impedance value preferably less than 5×10⁷ kg/m²-s, more preferably less than 4×10⁷ kg/m²-s and most preferably less than 2.5×10⁷ kg/m²-s. In one preferred embodiment of the present invention the vibrating components are comprised of aluminum alloys, titanium alloys, or combinations thereof, in order reduce acoustic power losses. In a preferred embodiment of the present invention, optional resonator 318 is made from high strength aluminum alloy, such as 2000, 6000, 7000 series alloys, or the like. In another embodiment of the present invention, driven wheel 310 is made from a material that is harder and more wear resistant than the material of optional resonator 308. In a preferred embodiment of the present invention driven wheel 310 is made from hardened steel, titanium alloy, brass, nickel alloy or ceramic.

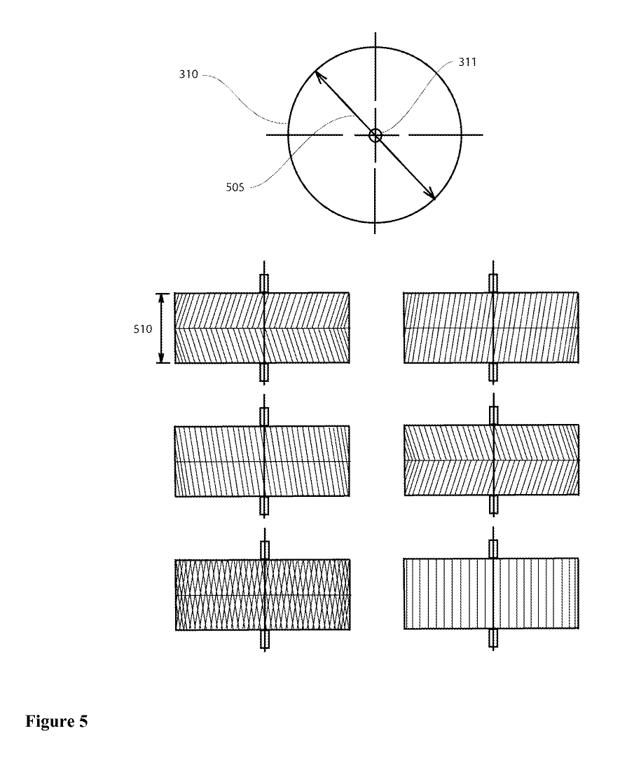

FIG. 5 shows some key features of driven wheel 310 in device 300. The diameter 505 of driven wheel 310 can be adjusted to achieve a wide range of output speed and torque values depending upon the specific medical task requirements of the output mechanical motion. According to one embodiment of the present invention, diameter 505 of driven wheel 310 is preferably between 0.2 cm and 20 cm, more preferably between 0.5 and 15.0 cm, and most preferably between 1.0 and 10.0 cm. The width 510 of driven wheel 310 is also an important design feature that is optimized according to the present invention, since increasing width 510 provides a larger region of contact with optional resonator 308, but also increases the overall size and mass of the driven member. According to one embodiment of the present invention, width 510 of driven wheel 310 is preferably between 0.1 cm and 10.0 cm, more preferably between 0.2 and 8.0 cm, and most preferably between 0.3 and 5.0 cm.

According to another embodiment of the present invention, the surface of the driven member is preferably modified in such a manner as to increase friction and decrease slippage at the region of contact between the frictional components. This may be accomplished by providing a surface having a non-smooth texture, for example through the use of non-smooth surface texture 418. There are numerous other methods known in the art for increasing frictional tractions between moving surfaces, and any of these methods may be utilized in devices of the present invention. For example, the surface of the driven member may be modified, treated or textured by machining, brushing, burnishing, knurling, sanding, roughening, grit blasting, or the application of surface coatings such as frictional coatings, abrasive coatings, and the like. FIG. 5 schematically illustrates several examples in which non-smooth surface texture 418 of driven wheel 310 in device 300 exhibits features that are produced as machined patterns with various combinations of grooves, knurls, teeth, bumps, ridges, and the like. Several design factors of these features may be adjusted to optimize the interfacial friction, power transfer efficiency, reliability and lifetime of the devices of the present invention, including but not limited to the shape, depth, area density, pitch, and angles of the features that comprise the textured surface.

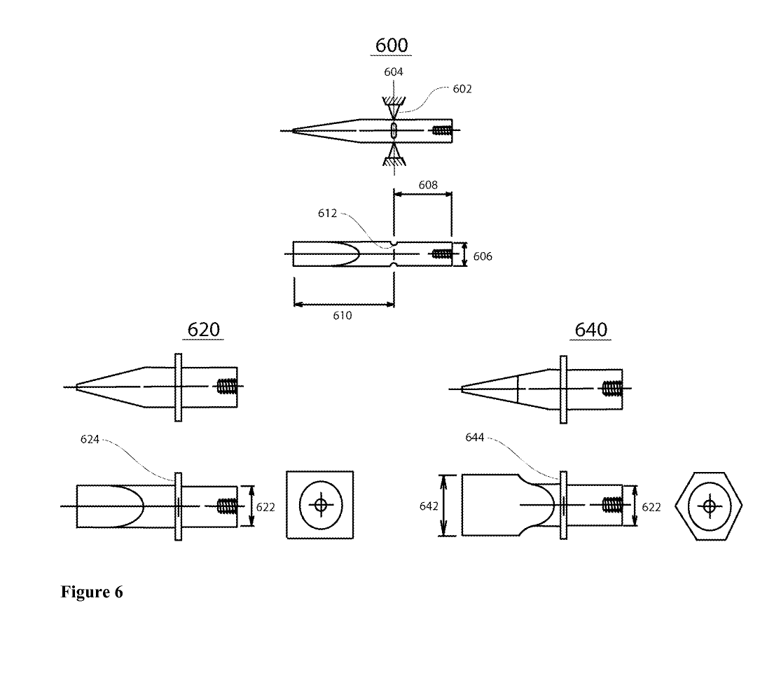

FIG. 6 schematically shows side and top views of optional resonator 308 in device 300. In one preferred embodiment of the present invention, optional resonator 600 is held in place by mechanical supports 602 at acoustic node 604, having diameter 606, distance 608 between the proximal end of optional resonator 600 and acoustic node 604, distance 610 between acoustic node 604 and the distal end of optional resonator 600. Note, the sum of distance 608 plus 610 equals the total length of the resonator, which must be tuned to match the operating frequency. Grooves 612 located at the position of acoustic node 604 facilitate proper positioning of mechanical supports 602. According to one embodiment of the present invention, diameter 606 is preferably between 0.1 cm and 10 cm, more preferably between 0.15 and 8 cm, and most preferably between 0.20 and 5.0 cm. Distance 608 is preferably between 0.5 cm and 10 cm, more preferably between 1.0 and 8.0 cm, and most preferably between 1.5 and 5.0 cm. Distance 610 is preferably between 0.5 cm and 20 cm, more preferably between 1.0 and 15.0 cm, and most preferably between 1.5 and 10.0 cm.

FIG. 6 further shows other examples of optional resonators according to the present invention. Optional resonator 620 is substantially similar to optional resonator 600, however, diameter 622 is increased compared to diameter 606 to provide a larger region of contact, and hence greater frictional traction, with driven wheel 310. Optional resonator 620 also comprises node flange 624 that provides an alternative method of supporting and affixing optional resonator 620 within device body 306, compared to pin-type node supports 406. Optional resonator 640 shows another possible configuration, where diameter 622 differs from distal end width 642. This affects the size of the region of contact with driven wheel 310, and additionally, by providing a greater or lesser mass of material within distance 610, a mechanism is provided for changing the amplitude of the acoustic displacements achieved at the distal end of optional resonator 640. For example, if cross sectional dimensions are held constant, distal end width 642 may either be larger (the example shown) or smaller (not shown) than diameter 622, increasing the region of contact while decreasing the amplitude of acoustic displacement, or decreasing the region of contact while increasing the amplitude of acoustic displacement, respectively. Optional resonator 640 also comprises node flange 644 that provides an alternative means of mechanical support. It should be obvious to those skilled in the art that various other types of node supports, such as rings, blades, clamps, flanges, and the like, may be used in devices of the present invention.

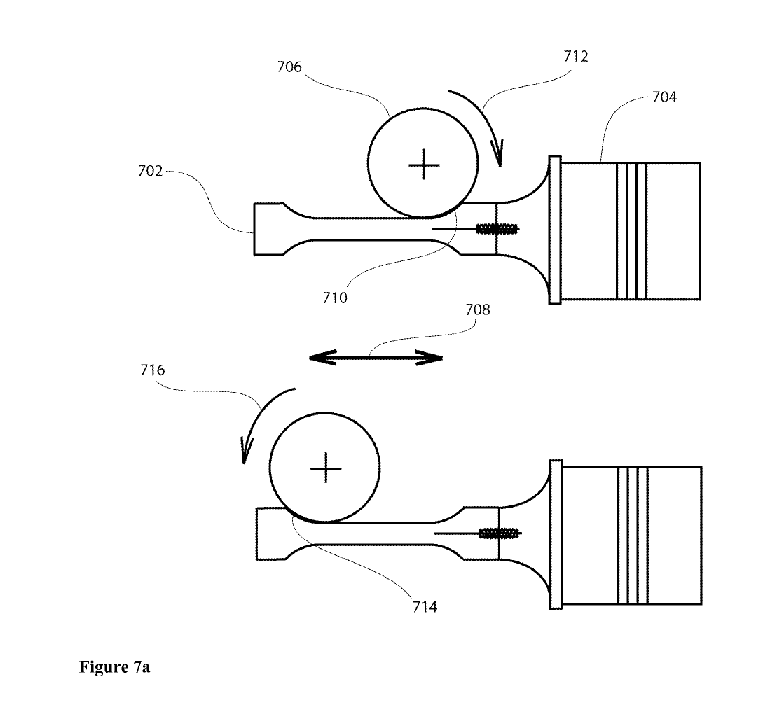

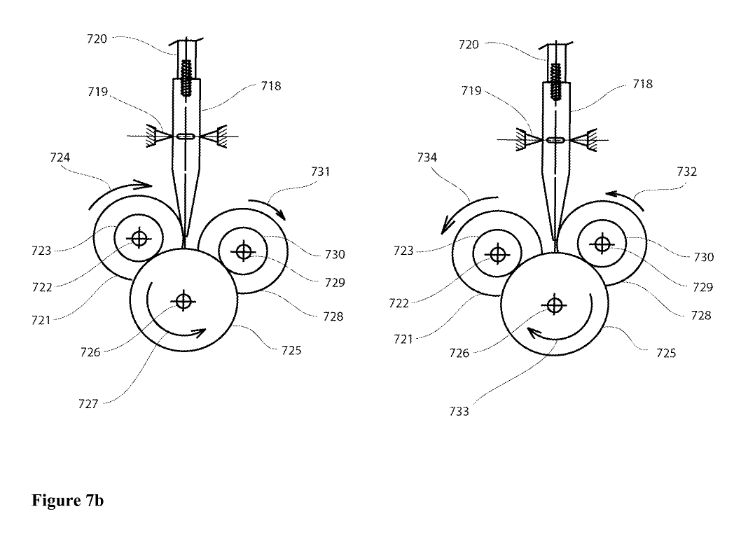

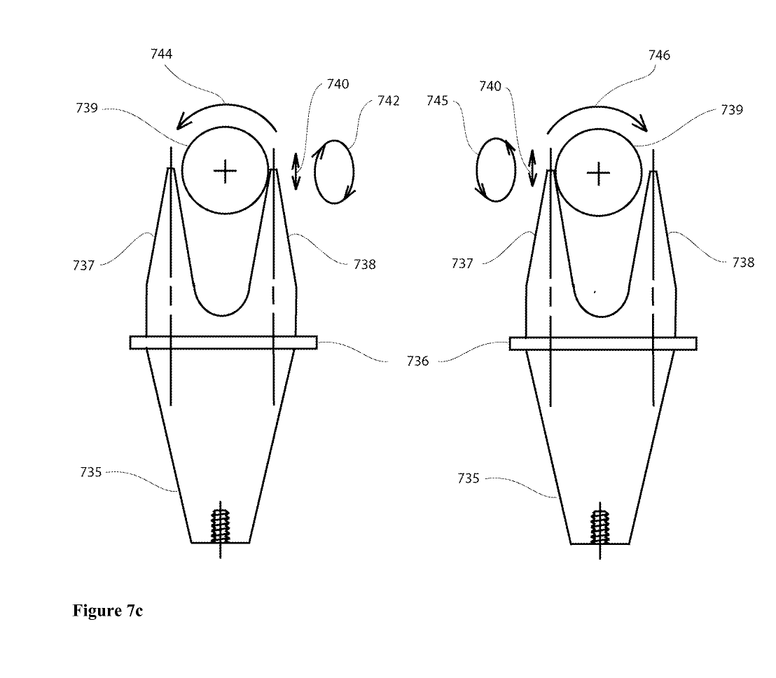

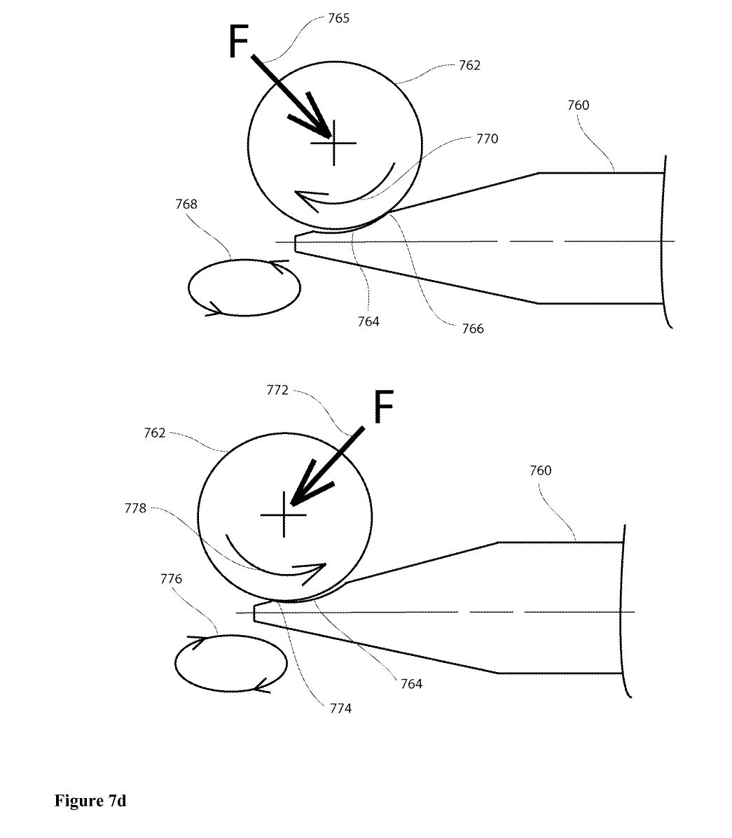

The devices of the present invention may further incorporate mechanisms and controlling means for generating both forward and reverse output motions, which can provide necessary or advantageous functionality for driving certain types of end-effectors. FIG. 7 shows several embodiments of the present invention where different mechanisms and controlling means for generating both forward and reverse motion capabilities are provided. In FIG. 7a, forward and reverse capability is provided as follows. Optional resonator 702 is matingly connected to transducer assembly 704 and is further in frictional contact with driven wheel 706. Optional resonator 702 and driven wheel 706 are capable of being repositioned relative to one another as shown at 708. Optional resonator 702 is further designed such that there are two different surfaces that may make contact with driven wheel 706. When driven wheel 706 is positioned in the rightmost position, frictional contact 710 is established and, when transducer assembly 704 is energized, driven wheel 706 rotates in a clockwise fashion 712. Alternatively, when driven wheel 706 is positioned in the leftmost position, frictional contact 714 is established and, when transducer assembly 704 is energized, driven wheel 706 rotates in a counterclockwise fashion 716.

FIG. 7b shows another embodiment of the present invention in which forward and reverse capability is achieved using two independent driven wheels that power a common output drive gear. Optional resonator 718 is supported by mechanical node supports 719 and is matingly connected to transducer assembly 720. In the configuration shown on the left, optional resonator 718 is brought into contact with a first driven wheel 721 that is fixedly mounted onto first shaft 722, along with first gear 723. Upon energizing, first driven wheel 721, first shaft 722 and first gear 723 are all caused to rotate clockwise 724. First gear 723 is engaged with output drive gear 725 that is mounted onto output drive shaft 726, both of which are therefore caused to rotate counterclockwise 727. The counterclockwise rotation 727 of output drive shaft 726 is a forward output mechanical motion. A second driven wheel 728 is fixedly mounted onto second shaft 729, along with second gear 730. Because second gear 730 is engaged with output drive gear 725, in the configuration shown on the left, second driven wheel 728 is caused to freely rotate clockwise 731. Alternatively, in the configuration shown on the right, optional resonator 718 is brought into contact with a second driven wheel 728 that is fixedly mounted onto second shaft 729, along with second gear 730. Upon energizing, second driven wheel 728, second shaft 729 and second gear 730 are all caused to rotate counterclockwise 732. Second driven wheel 728 is engaged with output drive gear 725 that is mounted onto output drive shaft 726, both of which are therefore caused to rotate counterclockwise 733. The counterclockwise rotation 733 of output drive shaft 726 is a reverse output mechanical motion. First driven wheel 721 that is fixedly mounted onto first shaft 722 along with first gear 723, and that is engaged with output drive gear 725 is caused to freely rotate counterclockwise 734. Thus, by providing a simple mechanical method (not shown) that moves the relative position of optional resonator 718 from being in contact with first driven wheel 721 to being in contact with second driven wheel 728, both forward and reverse output mechanical motion are achieved.

FIG. 7c shows another embodiment of the present invention in which forward and reverse capability is achieved by using an optional resonator that has two different tips, as follows. Optional resonator 735 is held in place by support flange 736 at the acoustic node, and has a left tip 737 and right tip 738, either of which may be brought into frictional contact with driven wheel 739. When energized by the transducer assembly (not shown), both the left tip 737 and right tip 738 vibrate as indicated by 740. As shown in the configuration on the left, when right tip 738 is brought into frictional contact with driven wheel 739, the tip produces elliptical oscillations 742 and driven wheel rotates counterclockwise 744, which is a forward output mechanical motion. Alternatively, when left tip 737 is brought into contact with driven wheel 739, the tip produces elliptical oscillations 745 and driven wheel 739 rotates clockwise 746, which is a reverse output mechanical motion. Therefore, by providing a simple mechanical means (not shown) of changing whether left tip 737 or right tip 738 makes frictional contact with driven wheel 739, both forward and reverse output mechanical motion are achieved.

FIG. 7d shows another embodiment of the present invention, similar to the mechanism of FIG. 7a, in which forward and reverse capability is achieved by changing the biasing force applied by the driven wheel onto the surface of the optional resonator, as follows. Optional resonator 760 is matingly connected to a transducer assembly (not shown) and is in frictional contact with driven wheel 762. Groove 764, having a circular profile substantially similar to the profile of driven wheel 762, exists near the tip of optional resonator 760. Groove 764 may preferably be formed in optional resonator 760 during its manufacture, or it may be produced or enlarged over time as a result of wear that occurs during use. In either case, when biasing force 765 causes driven wheel 762 to preferentially make contact with optional resonator 760 near the proximal end 766 of groove 764, then elliptical oscillations 768 occur as shown and driven wheel 762 rotates in a clockwise direction 770, which is a forward output mechanical motion. Alternatively, when biasing force 772 causes driven wheel 762 to preferentially make contact with optional resonator 760 near the distal end 774 of groove 764, then elliptical oscillations 776 occur as shown and driven wheel 762 rotates in a counterclockwise direction 778, which is a reverse output mechanical motion. Therefore, by providing a simple mechanical means (not shown) of changing the direction of the biasing force, and consequently the region of contact between driven wheel 762 and groove 764 in optional resonator 760, both forward and reverse output mechanical motion are achieved.

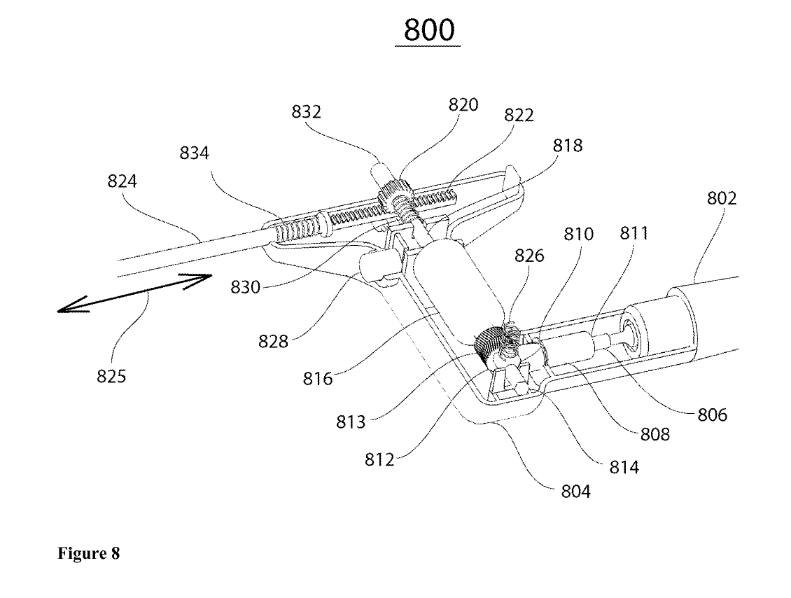

According to one embodiment of the present invention shown in FIG. 8, device 800 is a handheld appliance exemplary of configurations designed to produce relatively low speed, relatively high force linear output mechanical motion. Device 800 and substantially similar devices are preferred for driving end-effectors, or instrument attachments containing end-effectors, such as surgical staplers, surgical cutters, biopsy devices, suturing devices, clip appliers, and the like. Device 800 includes reusable handpiece 802 that removably attaches to device body 804, contains transducer assembly 806, and attaches to the power generator (not shown) via a cable (not shown). Optional resonator 808 is held within device body 804 by node support flange 810, and when handpiece 802 is attached to device body 804, the distal end of transducer assembly 806 matingly attaches to the proximal end of optional resonator via screw connection at 811. The distal end of optional resonator 808 is in frictional contact with driven wheel 812, that has knurled surface 813 and is fixedly attached to input drive shaft 814. Input drive shaft 814 connects to the input side of transmission assembly 816 that contains gear assembly (not shown) and slip clutch (not shown). The output side of transmission assembly 816 connects to output drive shaft 818 onto which output gear 820 is fixedly mounted and that engages linear rack 822. Working arm 824 is attached to the distal end of output rack 822, and is connected to, or provided as part of, the end-effector or instrument attachment further comprising the end-effector (not shown). Spring mechanism 826 controls the direction and magnitude of the force applied between optional resonator 808 and driven wheel 812. Trigger switch 828 allows hand activation and control of the device by variably adjusting the drive power supplied by the power generator to transducer assembly 806, and may be used in conjunction with or in place of a remote foot switch. Drive engagement spring 830 allows output rack 822 to be disengaged from output gear 820 when disengagement button 832 is depressed, thereby allowing output rack 822 and attached working arm 824 to be retracted to the starting position by retraction spring 834. This retraction mechanism provides a simple and fail-safe alternative to the forward and reverse output mechanical motion mechanisms described in FIG. 7, which may additionally be incorporated into device 800. Upon energizing the power generator and activation of trigger switch 828, the high frequency, low amplitude vibrations of transducer assembly 806 are transmitted to driven wheel 812 by optional resonator 808, which converts the vibrations into macroscopic rotary motion. Driven wheel 812 is a driven member, while input drive shaft 814, transmission assembly 816, output drive shaft 818, output gear 820 and linear rack 822 comprise a driven mechanism, which further converts the macroscopic rotary motion into linear output mechanical motion 825 having the desired speed, force and other characteristics. The linear output mechanical motion 825 is operatively transmitted to and drives the end-effector (not shown) in order to perform the intended medical function. For safety purposes, device 800 may also optionally include within the driven mechanism a slip clutch, mechanical fuse or other similar limiting feature known to those skilled in the art to prevent excessive speeds or forces from being generated.

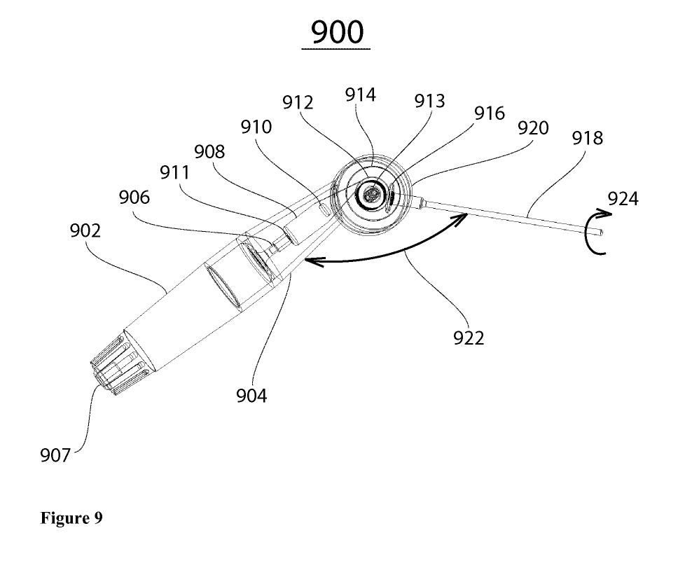

According to another embodiment of the present invention shown in FIG. 9, device 900 is a handheld appliance exemplary of device configurations designed to produce relatively high speed, relatively low torque rotary output mechanical motion. Device 900 and substantially similar devices are preferred for driving end-effectors, or instrument attachments containing end-effectors, such as surgical or dental drills, surgical or dental debriders, biopsy devices, tissue compactors, and the like. Device 900 includes handpiece 902 that removably attaches to device body 904, contains transducer assembly 906, and attaches to the power generator via cable connector 907. Optional resonator 908 is held within device body 904 by node support 910, and when handpiece 902 is attached to device body 904, the distal end of transducer assembly 906 matingly attaches to the proximal end of optional resonator via screw connection at 911. The distal end of optional resonator 908 is in frictional contact with driven wheel 912, that has a knurled surface (not shown) and is fixedly mounted on input drive shaft 913. Also fixedly mounted onto input drive shaft 913 is input gear 914 that engages directly with (i.e. without an intermediate transmission assembly) output gear 916, which is positioned at a 90 degree angle relative to input gear 914 and is fixedly mounted onto output drive shaft 918. Adjustably rotating housing 920 allows the relative angle 922 between input drive shaft 913 to output drive shaft 918 (and also handpiece 902) to be adjusted depending upon the needs of the medical procedure being performed. This user selectable articulating mechanism, whereby the end-effector is rotatable around an axis in order to change the orientation of the end-effector relative to the handpiece or driven member, provides significant advantages for certain types of medical procedures and is difficult to achieve in conventional powered devices. Upon energizing the power generator and activation of a trigger switch, foot switch, or the like (not shown), the high frequency, low amplitude vibrations of transducer assembly 906 are transmitted to driven wheel 912 by optional resonator 908, which converts the vibrations into macroscopic rotary motion. Driven wheel 912 is a driven member, while input drive shaft 913, input gear 914, output gear 916, and output drive shaft 918 comprise a driven mechanism, which further converts the macroscopic rotary motion into rotary output mechanical motion 924 having the desired speed, force and other characteristics. The rotary output mechanical motion 924 is operatively transmitted to the end-effector (not shown) in order to perform the intended medical function. Although not shown, forward and reverse mechanical motions may additionally be incorporated in device 900 according to the teachings of FIG. 7. For safety purposes, device 900 may optionally include within the driven mechanism a slip clutch, mechanical fuse or other similar limiting feature known to those skilled in the art to prevent excessive speeds or forces from being generated.

### EXAMPLES

### Example 1

An ultrasonic power system and ultrasonically powered device according to the present invention, configured to generate high force, linear output mechanical motion, as illustrated in FIG. 3, was constructed and tested as follows. A commercially available ultrasonic power generator operating at 55.5 kHz and rated for 75 watts maximum output power was connected to a commercially available plastic handpiece that contained an embedded ultrasonic transducer assembly. The embedded ultrasonic transducer assembly was designed to operate with said power generator such that during operation, longitudinal mechanical vibrations having an amplitude between 20 and 150 μm were produced at the distal tip of the transducer assembly, with increasing output power being user selectable by adjusting the output power selector switch between level 1 and level 5.

The handpiece was attached to a device body that was machined from Delrin™ plastic, into which was mounted an optional resonator, driven wheel, and moveable carriage assembly. The optional resonator had a threaded proximal end to accept and matingly attach to the transducer embedded within the handpiece. Said optional resonator was precision machined from 6061 aluminum alloy in the T6 heat treatment condition to have a total length that was generally in the range from 5.21 cm to 5.72 cm, and most optimally found to be between 5.33 cm and 5.59 cm. The optional resonator was fixedly mounted inside the device body using a flange support integrated into the optional resonator and having screw connections for mounting into the device body. The flange support was located at the position of an acoustic node, which was determined by experiment to be optimally located approximately 2.29 cm from the proximal end of said optional resonator.

The optional resonator was positioned in frictional contact along a surface near its distal tip with a hardened steel driven wheel approximately 1.59 cm in diameter and having a knurled surface texture produced by machining a series of angled grooves into its surface. The location of the region of contact between the optional resonator and the driven wheel was adjusted to the desired position by sliding forward or backward the portion of the device body to which the flange support was attached, thereby allowing the optional resonator to be positioned relative to the position of the driven wheel. For purposes of these experiments the moveable carriage assembly was positioned such that the angle of impingement between the driven wheel and the optional resonator was 0°. The force between the optional resonator and driven wheel was controlled and maintained constant by a steel spring attached at one end to the device body and at the other end to a moveable carriage assembly mounted on a pivoting shaft. The spring force was selected to be approximately 0.45 kg, resulting in a normal force being applied between the driven wheel and optional resonator of approximately 1.32 kg, taking into account the moment arm.

The driven wheel was fixedly mounted onto a 0.32 cm diameter rotating steel shaft held within the moveable carriage assembly, onto which was also fixedly mounted a drive gear 1.06 cm in diameter. The drive gear engaged a primary gear also 1.06 cm in diameter (gear ratio 1:1) mounted onto a 0.32 cm diameter primary drive shaft that extended out of the device body and into a transmission mounted onto the exterior of the device body using screw connections. The transmission consisted of a planetary gear assembly having an adjustable gear ratio, which for the purposes of these tests was selected to be either 20:1 or 100:1. The output shaft from the planetary gear assembly had an output gear approximately 0.95 cm in diameter fixedly mounted onto it, that was used to drive a 19 cm long linear steel rack. When the transducer was energized by the power generator, the driven wheel in frictional contact with the optional resonator was caused to rotate, said driven wheel rotation then being converted into linear motion by the driven mechanism and causing the linear rack to move in a forward direction.

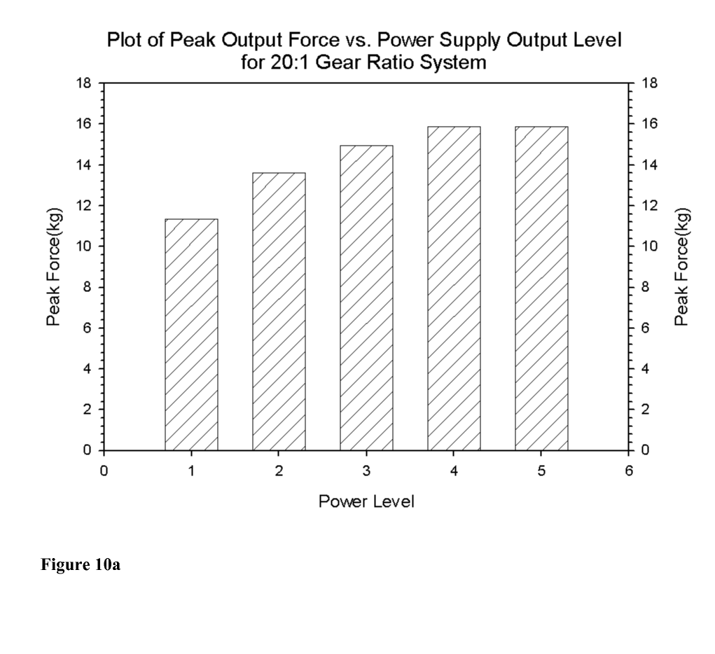

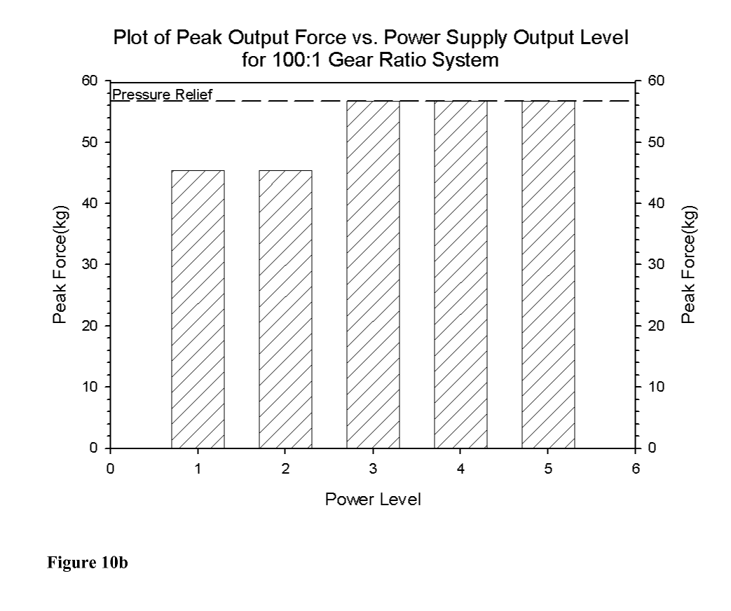

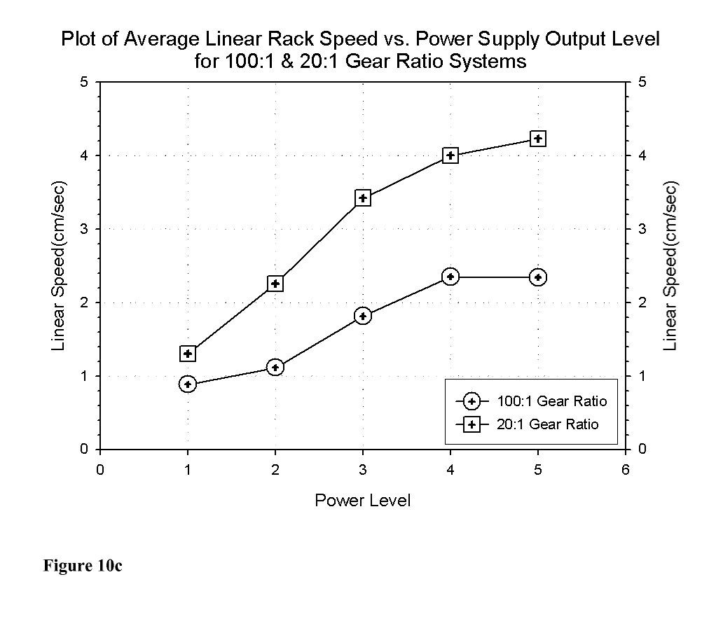

To measure the performance of the device, the device body was supported within a test fixture configured to hold a 5.08 cm diameter compressible air cylinder to which was connected a pressure gauge having a dial readout, thereby serving as an a prototype medical end-effector simulating a surgical stapler. By placing the distal end of the linear rack in contact with the proximal end of the piston on the air cylinder, and then energizing the device, the linear rack moved in a forward direction, pushing the piston, compressing the air within the air cylinder, and thereby causing the pressure to increase within the cylinder. The pressure within the cylinder was monitored over time by observing the dial gauge and recording the pressure reading. By knowing the cylinder diameter, the actual linear output force generated by the device was calculated. To prevent damage to the device from excessive forces, a pressure relief valve was used and was set to prevent the force from exceeding 56.8 kg. The maximum force during a particular experiment was taken to be the lesser of the force at which linear travel of the rack and piston stopped or the maximum allowable force of 56.8 kg set by the pressure relief valve. A stopwatch and calipers were used to measure the distance and speed of travel of the rack and piston during each test. Tests were performed at each of the 5 available power level settings on the power generator, for two different gear rations, 20:1 and 100:1. The results of these experiments are shown in FIG. 10.

FIG. 10a shows the maximum linear output force for the device at each of the 5 different power levels for the case when the planetary gear ratio was set at 20:1. The maximum linear output force for the device increased substantially linearly from approximately 11.4 kg to approximately 15.9 kg going from power level 1 to power level 4, then the force remained constant at 15.9 kg going from power level 4 to power level 5.

FIG. 10b shows the maximum linear output force for the device at each of the 5 different power levels for the case when the planetary gear ratio was increased to 100:1. The maximum linear output force for the device was approximately 45.5 kg for power level 1, however, at power levels 3-5 the maximum linear output force exceeded 56.8 kg, the maximum pressure allowed by the pressure relief valve.

While other device configurations are possible as described previously, the maximum linear output forces generated by both the 20:1 and 100:1 gear ratios in the functional prototype of Example 1 are significant and well suited for driving mechanical end-effectors for use in a wide variety of medical procedures. For example, these mechanical forces are sufficient to successfully perform a surgical stapling procedure.

FIG. 10c compares the linear speed of the device output at gear ratios of 20:1 and 100:1. For the 20:1 gear ratio, the linear speed of the device output increased from approximately 1.3 cm/s to approximately 4.2 cm/s as the power level increased from level 1 to level 5. For the 100:1 gear ratio, the linear speed of the device output increased from approximately 0.9 cm/s to approximately 2.4 cm/s as the power level increased from level 1 to level 4, however the speed remained constant going from power level 4 to power level 5. It is noteworthy from Example 1 that significant maximum linear output forces can be generated over a wide range of output speed, and this performance characteristic is potentially beneficial to surgeons when conducting certain medical procedures such as stapling or cutting.

### Example 2

An ultrasonic power system and ultrasonically powered device according to the present invention, configured to generate high speed rotary output mechanical motion was constructed and tested as follows. The device similar to that shown in FIG. 3 that was used in Example 1 was further modified by removing entirely the planetary gear assembly, output gear and linear rack. The drive shaft, onto which is fixedly mounted the primary gear, was extended through the device housing and supported by a bearing mounted in the wall of the device body. A coupler and drive shaft extension were used to lengthen the drive shaft, which then became the output shaft capable of rotary motion. Accordingly, when the driven wheel in frictional contact with the optional resonator was caused to rotate by energizing the transducer assembly, the rotary motion of the driven wheel was transferred directly through the drive gear mounted onto the same shaft as the driven wheel, to the primary gear mounted onto the drive shaft, that was then caused to rotate, producing a forward rotary output mechanical motion. The gear ratio between the drive gear and the primary gear could be adjusted in order to change the speed and force of the rotary output mechanical motion. In the series of tests described below, the gear ratio was selected to be 1:1. An output wheel was mounted onto the end of the drive shaft, and the diameter of the output wheel could be varied to further adjust the speed of the output mechanical motion. In the tests performed, the diameter of the output wheel was selected to be 1.27 cm.

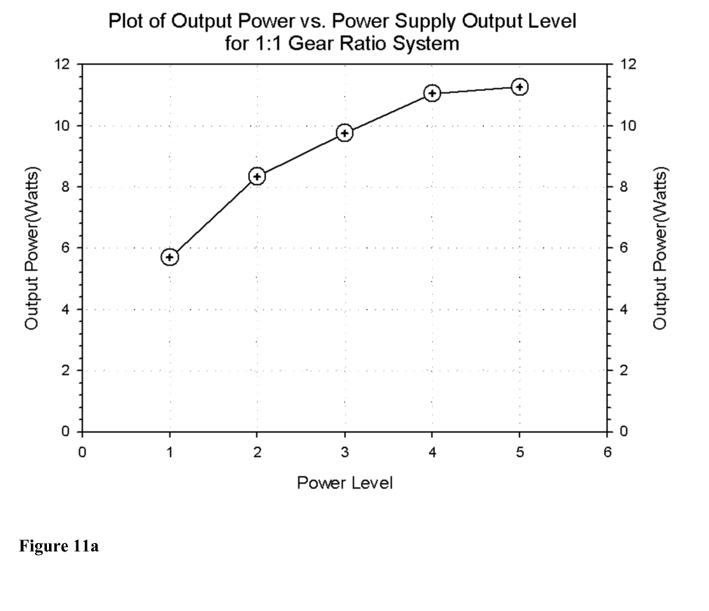

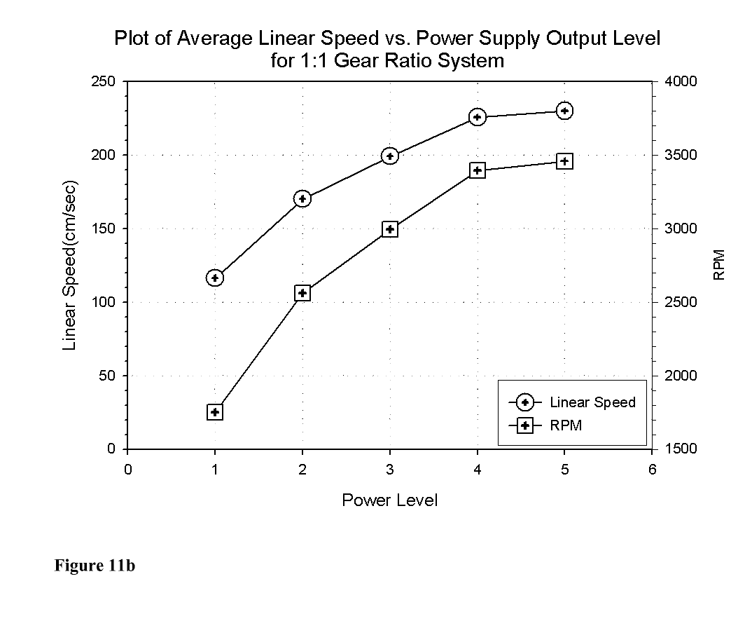

To measure the performance of the device, a string was attached to the output wheel and the device body was placed into a test fixture configured such that the output rotary motion was used to wind a string around the output wheel. A fixed weight of 0.25 kg was attached to the other end of the string, such that during operation, the drive shaft rotation caused the string to wind around the output wheel, thereby lifting the fixed weight against the force of gravity. This fixed weight and string configuration served as a prototype medical end-effector simulating a surgical or dental drill. A stopwatch and known length of the string were used to measure the distance and speed of travel during the test. Knowing the diameter of the output wheel, the fixed amount of weight lifted, and by calculating the speed, the output power was readily calculated. The tests were performed by varying the power level on the power generator from level 1 to level 5 and recording the linear speed generated by the output wheel. The results of these experiments are shown in FIG. 11.

FIG. 11a shows the output power of the device of Example 2 as a function of generator power level. The output power level increased continuously from approximately 5.7 watts to approximately 11.3 watts as the power level increased from level 1 to level 5, however it appears that at level 5 the output power may have been nearing a maximum for this device configuration. Output power information such as shown in FIG. 11a is useful in product design for understanding the types of medical procedures that may be successfully conducted for a given device configuration.

FIG. 11b shows the linear speed and revolutions per minute (rpm) generated by the device output. In direct correlation with the output power of FIG. 11a, the linear speed increased from approximately 116.3 cm/s to approximately 230.0 cm/s as the power level increased from level 1 to level 5. Similarly, the rpm increased from approximately 1750 to approximately 3460 as the power level increased from level 1 to level 5.

While other device configurations are possible as described previously, the rotary output power and speed generated the functional prototype of Example 2 are significant and well suited for driving mechanical end-effectors for use in a wide variety of medical procedures. For example, these power and speeds are sufficient to successfully perform a surgical drilling procedure

### Example 3

A device capable of forward and reverse linear motion according to the method shown in FIG. 7d was constructed and tested as follows. The device used in Example 1 was modified by first forming a noticeable groove into the optional resonator component near its distal tip at the region of contact with the driven wheel. The groove was formed to have a similar shape profile, but slightly larger radius of curvature compared to that of the driven wheel. The device body was also modified to allow the position of the moveable carriage assembly pivot point to be carefully adjusted relative to the position of the optional resonator. With proper positioning of the moveable carriage assembly, the direction of the applied force, as well as the location within the optional resonator groove where contact takes place (i.e. the proximal vs. distal face of the groove), could be altered simply by adequately increasing the force generated by the spring on the moveable carriage assembly, for example, by manually stretching the spring. In this way, with the force exerted by the carriage assembly spring in its normal configuration, driven wheel made contact was made on the proximal face of the groove in the optional resonator, the driven wheel turned in clockwise fashion and the linear rack moved in a forward direction. When the carriage assembly spring was manually stretched, the direction of force changed and driven wheel contact was made at the distal face of the groove in the optional resonator. In this case, the driven wheel turned in a counterclockwise fashion and the linear rack moved in a reverse direction. In this manner, simply by manually stretching and releasing the carriage assembly spring, the direction of travel of the linear rack could be reversed. The force and speed of the device output were confirmed to be comparable to the data presented for Example 1, and were approximately equivalent regardless of the direction of travel.

### REFERENCES

1. J. J. Vaitekunas et al, “Effects of Frequency on the Cutting Ability of an Ultrasonic Surgical Instrument,” 31st Annual Ultrasonic Industry Association Symposium, Oct. 11-12, 2001, Atlanta Ga.
2. http://www.nde-ed.org/index_flash.htm
3. J. Blitz and G. Simpson, “Ultrasonic Methods of Non-destructive Testing,” Springer, USA, 1996, ISBN0412604701.
4. http://ndeaa.jpl.nasa.gov/nasa-nde/usm/usm-hp.htm
5. S. Toshiiku and K. Takashi, “An Introduction to Ultrasonic Motors,” Oxford University Press, USA, 1994, ISBN0198563957.

I claim:

1) A medical device comprising:
a) At least one transducer capable of converting electrical energy into mechanical vibrations;
a) At least one driven member in frictional contact with said at least one transducer, wherein during operation of said device said frictional contact between said at least one transducer and said at least one driven member produces output rotary motion, linear motion, or combinations thereof; and
b) At least one end-effector driven by said output rotary motion, linear motion, and combinations thereof.

2) A device of claim 1 wherein said at least one transducer is selected from the group consisting of an ultrasonic transducer, a magnetostrictive transducer, and combinations thereof, wherein said ultrasonic transducer is selected from the group consisting of a standing wave-type ultrasonic transducer, a traveling wave-type ultrasonic transducer, and combinations thereof.

3) A device of claim 2 wherein said ultrasonic transducer produces mechanical vibrations having a frequency of vibration preferably greater than 1 kHz and amplitude of vibration preferably between 1 and 500 μm.

4) A device of claim 1 wherein said mechanical vibrations are selected from a group consisting of longitudinal vibrations, lateral vibrations, flexural vibrations, torsional vibrations, and combinations thereof.

5) A device of claim 1 wherein said frictional contact is selected from the group consisting of edge-type contact, surface-type contact, and combinations thereof.

6) A device of claim 1 further comprising at least one optional resonator matingly connected to said at least one transducer, wherein during operation of said device said optional resonator is in frictional contact with said at least one driven member.

7) A device of claim 6 wherein at least one portion of said at least one transducer or said at least one optional resonator that is in frictional contact with said at least one driven member is comprised of one or more materials having an acoustic impedance preferably less than 5×10⁷ kg/m²-s, and most preferably is comprised of one or more materials selected from the group consisting of aluminum alloys, titanium alloys, and combinations thereof.

8) A device of claim 6 wherein the interfacial friction and power transfer efficiency between the said at least one transducer or said at least one optional resonator and the said at least one driven member is substantially by providing a non-smooth texture on at least one portion of the surface of said at least one driven member.

9) A device of claim 6 wherein the interfacial friction and power transfer efficiency between the said at least one transducer or said at least one optional resonator and the said at least one driven member is substantially increased by providing at least one driven member surface comprising a material having a hardness greater than the material from which said at least one transducer or said at least one optional resonator is constructed.

10) A device of claim 1 additionally comprising at least one controller for adjusting at least one characteristic selected from the group consisting of the speed, force, and direction of said output motion, wherein said at least one controller is capable of adjusting said at least one characteristic in a manner selected from the group consisting of fixed adjustment, variable adjustment, and combinations thereof.

11) A device of claim 1 wherein the said at least one end-effector comprises a tool selected from the group consisting of a stapler, cutter, drill, compactor, debrider, biopsy sampler, suture former, clamper, clipper, spreader, extractor, and any combination of the foregoing.

12) A device of claim 1 further comprising at least one articulating mechanism that allows at least one portion of said end-effector to change orientation relative to the orientation of at least one orientation selected from the group consisting of the driven member orientation, the transducer orientation, and combinations thereof.

13) A device of claim 1 wherein said device is sterilized prior to use, is provided in a sterile package, and is intended for use on a single patient.

14) A device of claim 1 wherein at least one portion of said device is sterilized after initial use and is intended for use on one or more patients.

15) A medical device comprising:
a) A handle having at least one transducer capable of converting electrical energy into mechanical vibrations and a connector for electrically coupling the at least one transducer to a power generator;
b) At least one driven member in frictional contact with said at least one transducer, wherein during operation of said device said frictional contact between said at least one transducer and said at least one driven member produces output rotary motion, linear motion, and combinations thereof, and
c) At least one end-effector driven by said output rotary motion, linear motion, and combinations thereof.

16) A medical device for mounting on a handle having a transducer capable of converting electrical energy into mechanical vibrations, the device comprising:
a) At least one driven member positioned for contacting the transducer when the device is mounted on the handle and during vibration of the transducer, wherein said contact between said at least one driven member and the at least one transducer produces output rotary motion, linear motion, or combinations thereof, and
b) At least one end-effector driven by the output rotary motion, linear motion, or combinations thereof, wherein the end-effector is selected from the group consisting of a stapler, cutter, drill, compactor, debrider, biopsy sampler, suture former, clamper, clipper, spreader, extractor, and any combination of the foregoing.

17) A powered medical device system comprising:
a) A power generator;
b) At least one transducer capable of converting electrical energy into mechanical vibrations;
c) At least one driven member in frictional contact with said at least one transducer, wherein during operation of said device said frictional contact between said at least one transducer and said at least one driven member produces output rotary motion, linear motion, and combinations thereof, and
d) At least one end-effector driven by said output rotary motion, linear motion, and combinations thereof.

18) A system of claim 17 wherein said at least one end-effector further comprises one or more instrument attachments selected from the group consisting of a stapler, cutter, drill, compactor, debrider, biopsy sampler, suture former, clamper, clipper, spreader, extractor, and combinations of the foregoing, wherein said instrument attachments are optionally detachable and optionally interchangeable.

19) A system of claim 17 wherein said power generator is an ultrasonic generator comprising:
a) Energy output during operation having a frequency preferably greater than 1 kHz and power rating preferably greater than 1 Watt;
b) Optionally, at least one controller capable of at least one function selected from the group consisting of energizing, de-energizing, variably adjusting the power output delivered to said transducer, and any combination of the foregoing, wherein said controller is selected from the group consisting of hand switches, foot switches, wireless transmitter switches, voice activation switches, and combinations thereof.

20) A powered medical device system comprising:
a) A power generator;
b) A handle having at least one transducer capable of converting electrical energy into mechanical vibrations and a connector for electrically coupling the at least one transducer to said power generator;
c) At least one driven member in frictional contact with said at least one transducer, wherein during operation of said device said frictional contact between said at least one transducer and said at least one driven member produces output rotary motion, linear motion, or combinations thereof; and
d) At least one end-effector driven by said output rotary motion, linear motion, or combinations thereof.
